(19) United States
(12) Patent Application Publication
(10) Pub. No.: US 2006/0035932 A1
(43) Pub. Date: Feb. 16, 2006

(54) N-aryl piperidine compounds

(75) Inventors: Martin A. Murphy, St. Helena, CA (US); John Robert Schullek, Santa Clara, CA (US); John S. Ward, Redwood City, CA (US); Gary C. Look, Santa Clara, CA (US); Rama Jain, Fremont, CA (US); Laurence Lee, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Propharmacon, Inc., San Jose, CA (US)

(21) Appl. No.: 11/138,618

(22) Filed: May 25, 2005

Related U.S. Application Data
(60) Provisional application No. 60/575,282, filed on May 27, 2004.

(51) Int. Cl. A61K 31/4545; C07D 401/14; C07D 401/02

(52) U.S. Cl. 514/316; 546/186; 546/189

(57) ABSTRACT

The present invention provides N-aryl piperidine compounds having antiinfective activity (i.e., antibacterial, antiviral), their compositions and methods of use. The compounds of the invention prevent the entry of HIV into host cells. More particularly, the compounds are effective at in inhibiting the binding interaction of HIV glycoprotein gp120 and the cell surface receptor CD4. The compounds can be used alone or in combination with other antivirals, antiinfectives, immunomodulators, or HIV entry inhibitors. More particularly, the invention relates to the prevention and treatment of HIV infectivity and AIDS.

### CROSS REFERENCES TO RELATED APPLICATION

This application claims priority to U.S. Provisional Application No. 60/575,282, filed on May 27, 2004, the teaching of which is hereby incorporated by reference in its entirety for all purposes.

### FIELD OF THE INVENTION

The present invention provides substituted N-aryl-piperidines, their pharmaceutical compositions and methods of use. These compounds have been found to have HIV anti-viral activity. In particular, these compounds have been found to be especially useful in inhibiting the binding of glycoprotein 120 (gp 120) with CD4.

### BACKGROUND OF THE INVENTION

HIV-1 (human immunodeficiency virus-1) infection is a major medical concern, infecting an estimated 33.4 million people worldwide. An enormous amount of research has been done on HIV, resulting in the numerous drugs being currently prescribed. These fall into three general categories: nucleoside reverse transcriptase inhibitors (NRTI's), non-nucleoside reverse transcriptase inhibitors (nNRTI's), and protease inhibitors (PI's) (Gulick, R. M., Clin. Microbiol. Infect., 9(3), 186-93, 2003). Despite the success of these drugs at decreasing viral load, they do have significant toxicities associated with long-term use and also can pressure mutation of the virus to evolve resistant strains, thus rendering the drugs less effective. Therefore, there continues to be a significant, unmet medical need to combat the disease with new, effective drugs that are not cross-resistant to the current ones. In addition, because these drugs are not curative and must be taken for the duration of one's life, there is a dire need to develop drugs with little toxicity and side effects to ease suffering and enhance patient compliance in taking the medication.

With the above properties in mind, future-generation of compounds that target HIV viral replication are continually being evaluated and includes the NRTI, Entrivia™, developed by Gilead/Triangle Pharmaceuticals which has recently gained approval (Rousseau, F. S. et al., J. Antimicrob. Chemother., 48(4), 507-13, 2001; Wakeford C., Tenth Conference on Retroviruses and Opportunistic Infections, abstract, P550, Boston, 2003), a nNRTI, Amdoxovir, developed by Gilead is currently in phase II clinical trials (Mewshaw, J. P. et al., J. Acquir. Immune Defic. Syndr., 29(1), 11-20, 2002); Kessler, H., et al., Abstract and Poster Presentation TuPpA1146 at the XIIIth International AIDS Conference; July 9-14 (2000), Durban, South Africa), DPC-083, a NRTI that is in phase II clinical trials from Bristol Myers Squibb (Lin, P. F. et al., Proc. Natl. Acad. Sci. USA., 2003), and a protease inhibitor, Reyataz™, also from Bristol Myers Squibb which has recently been approved as a drug (Goldsmith, D. R. et al., Drugs., 63(16), 1679-95, 2003). With the significant problem of resistance developing to existing drugs, it is imperative to develop new classes of drugs for HIV therapy (Miller, R. H. et al., and AIDS Res. Hum. Retroviruses, 12(10), 859-865, 1996; O'Brien, W. A. AIDS Read., 13(3) Supply S4-8, 2003).

In addition to drugs that target viral replication, further scientific understanding of the mechanism of HIV entry into target cells has resulted in the discovery of a number of new potential targets for therapeutic intervention, specifically cell surface proteins and receptors that play a role in the regulating the binding and fusion events of the HIV viral infection cycle. Generally, the compounds that prevent HIV viral entry into the host cell are called “HIV entry inhibitors”. Among the different therapeutic approaches in development are inhibitors that could intervene during the one of the three processes of virion entry which is: attachment of the virus to host cells, followed by the interaction with co-receptors, and finally the fusion of the virus and the host cell membranes.

The CD4 receptor is a 55 Kd cell surface protein on helper T lymphocytes, which utilize MHC (Major Histocompability Complex) class II antigens during antigen-driven T-cell activation (Doyle, C., Strominger, J. L., Nature, 330, 256-259, 1987). The binding of CD4 to MHC class II stabilizes the interaction of the T-cell receptor, associated peptide antigen, and MHC. The CD4 receptor, therefore, plays a critical role in the recognition of self and immunity. It is therefore critical that a gp120/CD4 antagonist not also block the essential CD4/MHC interaction.

CD4 is composed of four extracellular immunoglobulin (Ig)-like domains, a transmembrane segment, and a short intracellular domain. CD4 has also been shown to serve as the receptor for the human immunodeficiency virus (HIV), the causative virus of AIDS (Sattentau, Q. J. et al., Cell, 52, 631-633, 1988). The initial binding of virus to T-cells is through the interaction of the envelope glycoprotein gp120 with the CD4 receptor. The HIV-1 envelope gene (env) codes for the envelope glycoprotein precursor, gp160, which is proteolytically cleaved to yield gp120 and a membrane-spanning glycoprotein gp41, which are non-covalently associated with one another on the membrane surface. There are several lines of evidence showing the major route of infection of cells by the virus is by its binding to CD4 and therefore, HIV predominantly infects CD4+ cells, although a second avenue of infection in dendritic cells appears to not utilize CD4 but rather endocytosis is known (Engering, A. et al., J. Immunol., 168, 2118-26, 2002). It is known there is a correlation of a decrease in the CD4+ cells with the onset of AIDS (Rowland-Jones, S., Lancet, 354, 5-7, 1999). Taken together the current data supports the notion that the CD4/gp120 interaction is essential for HIV infection.

A great deal is known about the structural mechanisms of the CD4 interactions. The binding of MHC class II receptor and gp120 both utilize the N-terminal Ig domain 1 of CD4 (Wang, J. H. et al., PNAS, 98, 10799-10804, 2001). The crystal structure of the first two domains of CD4 has been known for some time. Crystallographic structures have been elucidated for both MHC II/CD4 and gp120 in complex with CD4 and a neutralizing antibody (Kwong, P. D. et al., Nature, 393, 648-659). These studies define a common binding region of the CD4 protein used for the two interactions.

Following the binding of gp120 to cell surface CD4, a conformational change occurs in the gp120 that allows it to bind to other membrane co-receptors which ultimately leads to fusion of viral and target cell membranes with introduction of the viral genome into the cell cytoplasm. Evidence suggests gp41 is the fusion component of the envelope glycoprotein (Kilby, J. M., Nat. Med., 4, 1302-1307, 1998). The co-receptor proteins, CXCR4 and CCR5, which have been shown to be required for fusion (Berger, E. A. et al., Annu. Rev. Immunol., 17, 657-700, 1999) belong to the chemokine receptor family and function in the later events of the fusion process.

Among the different therapeutic approaches possible are the development of inhibitors that could intervene during the three step process of virion entry which are: attachment of the virus to host cells, followed by the interaction with co-receptors, and finally the fusion of the virus and the host cell membranes. Fuzeon™ (T-20) a prototypic fusion inhibitor, which is a 36 amino acid peptide targeting gp4l was recently approved for use and is being commercialized by Trimeris/Hofmann-LaRoche. It is the only drug of this class approved to date (Lazzarin, A. et al., N. Engl. J. Med., 348(22), 2186-95, 2003). Although it is a twice-daily injectable, it may find its greatest use in individuals who demonstrate resistance to current therapies since it is likely to not be cross-resistant. A small molecule CCR5 inhibitor called SCH-C from Schering Plough is in preclinical development (Tsamis, F. et al., J. Virol., 77(9), 5201-8, 2003). Another small molecule inhibitor in late preclinical development is a CCR5 inhibitor, TAK 779 (Takashima, K. et al., Antimicrob. Agents Chemother., 45(12), 3538-43, 2001), and Progenics Pharmaceuticals is also developing a CCR5 antagonist.

Given the essential role of the CD4 receptor for early HIV attachment, intervention at this step would serve as a logical target for anti-viral therapy. Currently, there are a few early attachment inhibitors which have advanced to near approval status and these would include compounds such as PRO-542 which is a hybrid protein injectable in phase I/II development (Ketas, T. J. et al., J. Virol., 77(4), 2762-7, 2003), BMS-806 which is a small molecule preclinical lead which directly binds gp120. Cosalane, aurintricarboxylic acid, dextran sulfate, and PRO-2000 have been discontinued as oral therapeutics.

In addition to creating novel drugs for combating the disease, new avenues of drug use such as the creation of topical microbicides that can be used by women for their own personal protection as well as their sexual partner is one avenue being investigated to prevent the spread of HIV. To date, there is no marketed topical microbicide available for vaginal or rectal use for the prevention of HIV.

Currently, there are on-going programs seeking to develop a topical microbicide. PRO2000™ being developed by Indevus Pharmaceuticals is currently the only clinical candidate and is a sulfonic acid polymeric condensate. Cyanovirin, a protein isolated from a blue-green alga that interacts with gp120 (Boyd, M. R. et al., Antimicrob. Agents Chemother., 41(7), 1521-1530, 1997) is in preclinical development at Biosyn.

In the treatment of HIV and AIDS, there continues to be a significant, unmet medical need to combat the disease with new, effective drugs that are not cross-resistant to the current therapies. Moreover, in view of the understanding of the role of gp120/CD4 binding in HIV viral entry, there is a need in the art for new more effective compounds that are inhibitors of the gp120/CD4 interaction. These compounds can then be used to treat or prevent HIV infectivity. The present invention provides such new compounds, compositions and methods of treatment.

### BRIEF SUMMARY OF THE INVENTION

The present invention provides substituted N-aryl-piperidines that are effective inhibitors of the gp120 and CD4 binding interaction. As such, the present invention provides substituted N-aryl-piperidines having the general structure: 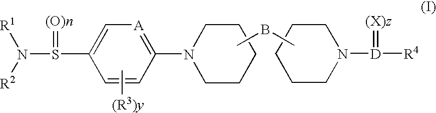
wherein R¹ and R² are each independently a member selected from the group consisting of hydrogen, alkyl, cycloalkyl, heterocycloalkyl, arylalkyl, heteroalkyl, heterocycloalkyl, heterocycloalkylalkyl, heteroarylalkyl and —CH₂CO₂H. Alternatively, R¹ and R² together with the nitrogen atom to which they are attached, form a 4- to 7-membered heterocyclic ring optionally containing 1-3 additional heteroatoms as ring members and optionally substituted with substituents selected from the group consisting of alkyl, heteroalkyl, aryl, heteroaryl, hydroxy, halogen, —CO₂R¹¹, wherein R¹¹ is hydrogen or (C₁-C₄)alkyl and alkoxy, wherein if present, any of said substituents located on adjacent atoms in said 4- to 7-membered ring may optionally be replaced with a substituent of formula -E-(CH₂)ᵤ—F— to form a fused ring wherein u is an integer from 1-2 and E and F are each independently CH₂, O or NH. Up to three bonds in the fused ring formed may optionally be replaced with a double bond; and wherein the fused ring formed may further be substituted with 0-4 substitutents selected from the group consisting of halogen, haloalkyl, alkyl, aryl, —CN and —NO₂. R³ is a member selected from the group consisting of halogen, alkyl, heteroalkyl, halo(C₁-C₄)alkyl, —S(O)₂R¹³, —S(O)R¹³ and —NO₂, wherein R¹³ is selected from the group consisting of (C₁-C₄)alkyl, alkylamino and amino. The symbols n and z may be the same or different and are integers from 1-2. Y is an integer from 0-4. A is N or C. B is (C₁-C₁₀)alkylene or (C₁-C₁₀)heteroalkylene. D is C or S. X, if present, is O, N or S. R⁴ is selected from the group consisting of aryl and heteroaryl. These compounds have activity as inhibitors of the gp120/CD4 binding interaction.

In other aspects, this invention provides pharmaceutical compositions of the compounds of formula I or a pharmaceutically acceptable salt thereof with a pharmaceutically acceptable carrier.

The compounds of formula I are useful in the treatment of viral and bacterial infections, in particular for the treatment and prevention of HIV infection and AIDS (Acquired Immune Deficiency Syndrome). As such, the present invention also provides a method of treating or preventing HIV infection or AIDS comprising administering to a subject having HIV infection or AIDS with an effective amount of a compound of formula I. These and other aspects, advantages and embodiments will become more apparent in the detailed description which follows.

### BRIEF DESCRIPTION OF THE DRAWINGS

FIG. 1 provides a general synthetic scheme for making the compound of the present invention.

### DETAILED DESCRIPTION OF THE INVENTION

I. Definitions

The term “alkyl,” by itself or as part of another substituent, means, unless otherwise stated, a straight or branched chain hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms, with those groups having 10 or fewer carbon atoms being preferred in the present invention.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1 -piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon substituent which can be a single ring or multiple rings (up to three rings) which are fused together or linked covalently. Non-limiting examples of aryl groups include phenyl, 1-naphthyl, 2-naphthyl, 4-biphenyl and 9-anthracenyl.

The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Substituents for each of the above noted aryl and heteroaryl ring systems are selected from the group of acceptable substituents described below.

The term “aryl” or “heteroaryl” when used in combination with other terms (e.g., aryloxy, arylthioxy, arylalkyl, heteroarylalkyl) is meant to include those radicals in which an aryl or heteroaryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: -OR′, ═O, ═NR′, ═NOR′, —NR′R″, —SR′, —halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2m′+1), where m′ is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, heteroaryl substituted with 1-3 halogens, unsubstituted alkyl, alkoxy or thioalkoxy groups, or aryl-(C₁-C₄)alkyl groups. When R′ and R″ or R″ and R″′ are attached to the same nitrogen atom, they can be combined with the nitrogen atom to form an optionally substituted 4-, 5-, 6-, or 7-membered ring. For example, —NR′R″ is meant to include 1-pyrrolidinyl and 4-morpholinyl. Optionally the newly formed 4- to 7-membered ring may additionally be substituted with a variety of group selected from: alkyl, heteroalkyl, aryl, heteroaryl, hydroxyl, halogen and alkoxy. Alternatively, two adjacent substitutents on the newly formed 4- to 7-membered ring may be replaced with a substitutuent of formula -E-(CH₂)ᵤ—F—, where u is 1-2, E and F are independently CH₂, and where up to 3 bonds in the new ring formed may optionally be replaced with a double bond and where the new ring formed may further be substituted with 0-4 substituents selected from the group consisting of halogen, halo(C₁-C₄)alkyl, alkyl, —CN and —NO₂. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl and heteroaryl groups are varied and are selected from: —halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R″′, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R″′ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ-X-(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

As used herein, the term “treating” a disease condition, includes either treating the disease in its active or remissive state as well as preventing or delaying the onset or contraction of the disease condition.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, “HIV reverse transcriptase inhibitor” is intended to refer to both nucleoside and non-nucleoside inhibitors of HIV reverse transcriptase (RT). Examples of nucleoside RT inhibitors include, but are not limited to, zidovudine (AZT, Retrovir®), zalcibidine (Hivid®, ddC), didanosine (Videx®, ddI), stauvidine (Zerit®, d4T), and lamivudine (Epivir®, 3TC). Examples of non-nucleoside RT inhibitors include, but are no limited to, delavirdine (Rescriptor®), efavirenz (Sustiva®) and nevirapine (Viramune®).

As used herein, “HIV protease inhibitor” is intended to refer to compounds which inhibit HIV protease. Examples include, but are not limited, saquinavir (Fortovase®), ritonavir (Norvir®), indinavir (Crixivan®), amprenavir (Agenerase®), nelfinavir (Viracept®).

As used herein, “HIV entry inhibitor” is intended to refer to compounds which prevent the entry of HIV into a host cell. Examples include but are not limited to, Fuzeon® and SCH-C.

By “therapeutically effective amount” herein is meant the amount of the subject compound that will elicit the biological or medical response of a tissue, system, animal or human that is being sought by the researcher, veterinarian, medical doctor or other clinician. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

As used herein, the term “protecting group” refers to a grouping of atoms that when attached to a reactive group in a molecule masks, reduces or prevents that reactivity. Examples of protecting groups can be found in T. W. Greene and P. G. Wuts, Protective Groups in Organic Chemistry, (Wiley, 2nd ed. 1991) and Harrison and Harrison et al., Compendium of Synthetic Organic Methods, Vols. 1-8 (John Wiley and Sons, 1971-1996). Representative amino acid protecting groups include formyl, acetyl, trifluoroacetyl, benzyl, benzyloxycarbonyl (CBZ), tert-butoxycarbonyl (BOC), trimethylsilyl (TMS), 2-trimethylsilyl-ethanesulfonyl (SES), trityl and substituted trityl groups, alloxycarbonyl, 9-fluorenylmethyloxycarbonyl (FMOC), nitro-veratryloxycarbonyl (NVOC) and the like. Representative hydroxy protecting groups include those where the hydroxyl group is either acylated or alkylated such as benzyl and trityl ethers as well as alkyl ether, tetrahydropyranyl ethers, trialkylsilyl ethers and allyl ethers.

II. Description of the Embodiments

A. Compounds

The present invention provides compounds which are useful as anti-infective agents. In particular, the present invention provides substituted N-aryl-piperidines having the formula:

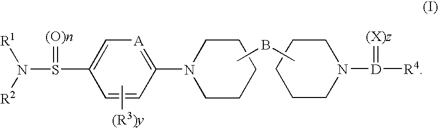

In formula I, the substituents R¹ and R² are each independently a member selected from the group consisting of hydrogen, alkyl, cycloalkyl, heterocycloalkyl, arylalkyl, heteroalkyl, heterocycloalkyl, heterocycloalkylalkyl, heteroarylalkyl and —CH₂CO2H. Optionally R¹ and R² can be taken together with the nitrogen atom to which they are attached join to form a 4- to 7-membered heterocyclic ring optionally having additional heteroatoms as ring members. This 4- to 7-membered ring can further be substituted with substituents selected from the group consisting of alkyl, heteroalkyl, aryl, heteroaryl, hydroxy, halogen, —CO₂R¹¹, wherein R¹¹ is hydrogen or (C₁-C₄)alkyl, and alkoxy. Preferably, the 4- to 7-membered ring is also substituted with a —CO₂R¹¹ group in which R¹¹ is hydrogen or (C₁-C₄)alkyl. More preferably, the 4- to 7-membered ring is substituted with a —CO₂H group. Any of the substituents located on adjacent atoms in the above 4- to 7-membered ring may optionally be replaced with a substituent of formula -E(CH₂)ᵤF— to form a fused ring wherein, u is an integer from 1-2, and E and F are each independently CH₂, O or NH. Up to three bonds in the fused ring formed may optionally be replaced with a double bond and the fused ring formed may further be substituted with 0-4 substitutents selected from the group consisting of halogen, haloalkyl, alkyl, aryl, —CN and —NO₂.

Preferably, the [symbol] —NR¹R² moiety in formula I is selected from one of the following groups:

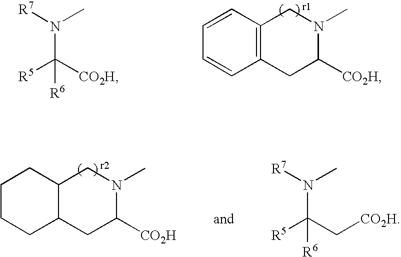

In formula I, R⁵, R⁶ and R⁷ are independently selected from the group consisting of hydrogen, alkyl, cycloalkyl, heteroalkyl, aryl, heteroaryl, arylalkyl, heteroarylalkyl. In one preferred embodiment, R⁵ and R⁶ are hydrogen, heteroalkyl or arylalkyl. In another preferred embodiment, the R⁵ and R⁶ substituents are each independently hydrogen, arylmethyl, heteroarylmethyl, or —(CH₂)ₙNHR in which n is 3 or 4, R is selected from the group consisting of: —C(O)R¹², —C(O)OR¹², and —S(O)₂R¹² wherein R¹² is either (C₁-C₆)alkyl, aryl, heteroaryl, benzyl or heteroalkyl. In another preferred embodiment, R⁵ and R⁶ or R⁵ and R⁷ together with the atom(s) to which they are attached form a 4- to 7-membered ring optionally having 1-3 heteroatoms as ring members, wherein said 4- to 7-membered ring is optionally substituted with 1-4 substituents selected from the group consisting of alkyl, heteroalkyl, aryl, heteroaryl, hydroxy, halogen, —C(O)₂H and alkoxy. Any of the substituents located on adjacent atoms in the 4- to 7-membered ring may optionally be replaced with a substituent of formula -E-(CH₂)ᵤ—F— to form a new ring in which u is an integer from 1-2, and E and F are each independently CH₂. Additionally, up to 3 bonds in the fused ring formed may optionally be replaced with a double bond. Furthermore, the fused ring formed may further be substituted with 0-4 substituents selected from the group consisting of halogen, halo(C₁-C₄)alkyl, alkyl, —CN and —NO₂. r1 and r2 are each an integer from 0-1.

In formula I, the variables n and z can be the same or different and are each an integer from 1-2 and y is an integer from 0-4. Preferably, n is an integer between 1-2 and y is an integer from 1-2.

In formula I, R³ is a member selected from the group consisting of halogen, alkyl, heteroalkyl, halo(C₁-C₄)alkyl, —S(O)₂R¹³, —S(O)R¹³ and —NO₂, wherein R¹³ is selected from the group consisting of (C₁-C₄)alkyl, alkylamino and amino. Preferably, the R³ substituted with halogen or halo(C₁-C₄)alkyl group. More preferably, R³ substituent is chloro, bromo or a trifluoromethyl group.

A, in formula I, represents a nitrogen or carbon atom, B represents a linker that is either a (C₁-C₁₀)alkylene or (C₁-C₁₀)heteroalkylene group, and D represents a carbon or sulfur atom. More preferably, B is a (C₂-C₇)alkylene or (C₂-C₇)heteroalkylene linker and D is a sulfur atom.

X is O, N or S. Preferably X is an oxygen atom. R⁴ represents an aryl or heteroaryl group. Preferably, R⁴ is a phenyl, a 2-thienyl, a 3-thienyl, a 2-pyridyl, a 3-pyridyl, or a 4-pyridyl group.

In another embodiment, the compound of the present invention has formula II 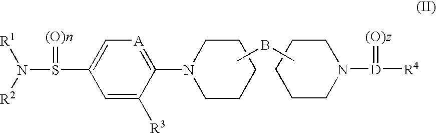
wherein R¹, R², R³, R⁴, A, B, D, n and z are as defined above.

In one embodiment, in formula II, the [symbol] -NR¹R² group is one of the following:

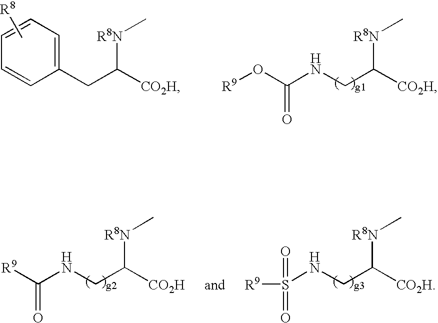

R⁸, in formula II, represents either a hydrogen atom, a halogen atom, an alkyl, heteroalkyl, aryl, heteroaryl, arylalkyl, heteroarylalkyl, halo(C₁-C₄)alkyl, hydroxyl, alkoxy or —NO₂ group; the subscripts g1 to g3 are each an integer from 1-4; and the symbol R⁹ represents an alkyl, heteroalkyl, aryl, heteroaryl, arylalkyl, heteroarylalkyl or halo(C₁-C₄)alkyl group.

More preferably, in formula II, R⁴ is selected from one of the following groups:

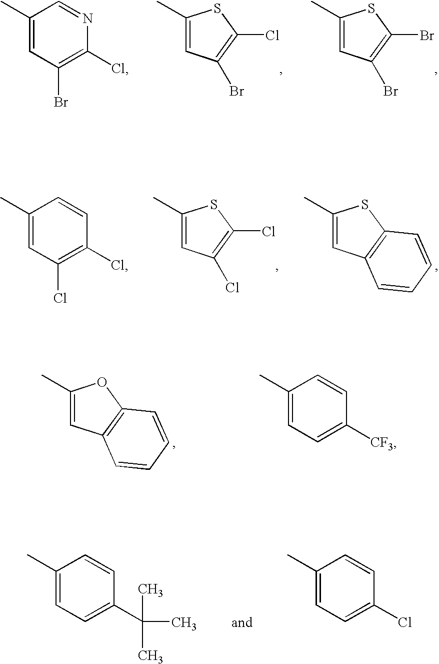

A family of specific compounds of particular interest within Formula I consists of compound and pharmaceutically acceptable salts thereof as set forth in Table I.

### Table I

1. 2-(3′-Bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonylamino)-3-phenyl-propionic acid

2. 2-(3′-Bromo-4-{6-[1-(4-bromo-5-chloro-thiophene-2-sulfonyl)-piperidin-4-yl]-hexyl}-3 ,4,5,6-tetrahydro-2H-[1,2′]bipyridinyl-5′-sulfonylamino)-3-phenyl-propionic acid

3. 2-(4-{5-[1-(4-Bromo-5-chloro-thiophene-2-sulfonyl)-piperidin-4-yl]-pentyl}-3′-chloro-3,4,5,6-tetrahydro-2H-[1,2′]bipyridinyl-5′-sulfonylamino)-3-phenyl-propionic acid

4. 2-(3′-Bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonyl)-1,2,3,4-tetrahydro-isoquinoline-3-carboxylic acid

5. 2-(3′-Bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonylamino)-3-phenyl-propionic acid

6. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(4-chloro-phenyl)-propionic acid

7. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-6-(2-chloro-benzyloxycarbonylamino)-hexanoic acid

8. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-pentafluorophenyl-propionic acid

9. {(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-[2-(4-fluoro-phenyl)-ethyl]-amino}-acetic acid

10. 1-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-4-phenyl-piperidine-4-carboxylic acid

11. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(3,4,5-trifluorophenyl)-propionic acid

12. 1-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-2,3-dihydro-1H-indole-2-carboxylic acid

13. (3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-cyclohexyl-acetic acid

14. 2-(3′-Bromo-4-{4-[1-(4-bromo-5-chloro-thiophene-2-sulfonyl)-piperidin-4-yl]-butyl}-3,4,5,6-tetrahydro-2H-[1,2′]bipyridinyl-5′-sulfonyl)-1,2,3,4-tetrahydro-isoquinoline-3-carboxylic acid

15. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-6-(4-bromo-5-chloro-thiophene-2-sulfonylamino)-hexanoic acid

16. 3-(3′-Bromo-4-{4-[1-(4-bromo-5-chloro-thiophene-2-sulfonyl)-piperidin-4-yl]-butyl}-3,4,5,6-tetrahydro-2H-[1,2′]bipyridinyl-5′-sulfonylamino)-3-phenyl-propionic acid

17. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-m-tolyl-propionic acid

18. [(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-cyclohexyl-amino]-acetic acid

19. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-6-(4-methyl-benzoylamino)-hexanoic acid

20. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(4-nitro-phenyl)-propionic acid

21. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-1,2,3,4-tetrahydro-isoquinoline-3-carboxylic acid

22. 6-Benzoylamino-2-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-hexanoic acid

23. 2-(3′-Bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonylamino)-3-p-tolyl-propionic acid

24. 1-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-octahydro-indole-2-carboxylic acid

25. [2-(3′-Bromo-4-{4-[1-(4-bromo-5-chloro-thiophene-2-sulfonyl)-piperidin-4-yl]-butyl}-3 ,4,5,6-tetrahydro-2H-[1,2′]bipyridinyl-5′-sulfonyl)-1,2,3,4-tetrahydro-isoquinolin-3-yl]-acetic acid

26. 2-(4-{5-[1-(4-Bromo-5-chloro-thiophene-2-sulfonyl)-piperidin-4-yl]-pentyl}-3′-trifluoromethyl-3,4,5,6-tetrahydro-2H-[1,2′]bipyridinyl-5′-sulfonylamino)-3-phenyl-propionic acid

27. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-(4-fluoro-phenyl)-propionic acid

28. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(4-bromophenyl)-propionic acid

29. 2-(3′-Bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonylamino)-4-methylsulfanylbutyric acid

30. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-6-(4-methoxybenzoylamino)-hexanoic acid

31. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-ethylsulfanylbutyric acid

32. [2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-1,2,3,4-tetrahydro-isoquinolin-3-yl]-acetic acid

33. 6-(4-Bromo-benzoylamino)-2-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-hexanoic acid

34. 2-(3′-Bromo-4-{4-[1-(4-bromo-5-chloro-thiophene-2-sulfonyl)-piperidin-4-yl]-butyl}-3,4,5,6-tetrahydro-2H-[1,2′]bipyridinyl-5′-sulfonylamino)-3-phenyl-propionic acid

35. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(3-chlorophenyl)-propionic acid

36. 5-Benzyloxycarbonylamino-2-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-pentanoic acid

37. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-6-(2,4-difluorobenzoylamino)-hexanoic acid

38. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-furan-2-yl-propionic acid

39. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-p-tolyl-propionic acid

40. 3-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-phenyl-propionic acid

41. 2-(3′-Bromo-4-{4-[1-(4-bromo-5-chloro-thiophene-2-sulfonyl)-piperidin-4-yl]-butyl}-3,4,5,6-tetrahydro-2H-[1,2′]bipyridinyl-5′-sulfonylamino)-3-thiophen-2-yl-propionic acid

42. 2-(4-{5-[1-(4,5-Dibromo-thiophene-2-sulfonyl)-piperidin-4-yl]-pentyl}-3′-trifluoromethyl-3,4,5,6-tetrahydro-2H-[1,2′]bipyridinyl-5′-sulfonylamino)-3-phenylpropionic acid

43. 3-[4-(4-{4-[1-(4-Bromo-5-chloro-thiophene-2-sulfonyl)-piperidin-4-yl]-butyl}-piperidin-1-yl)-3-chloro-benzenesulfonylamino]-cyclopentanecarboxylic acid

44. 1-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-5-phenyl-pyrrolidine-2-carboxylic acid

45. 2-(3′-Bromo-4-{6-[1-(4-bromo-5-chloro-thiophene-2-sulfonyl)-piperidin-4-yl]-hexyl}-3,4,5,6-tetrahydro-2H-[1,2′]bipyridinyl-5′-sulfonylamino)-4-methylsulfanylbutyric acid

46. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-(3,5-difluoro-phenyl)-propionic acid

47. 2-(3′-Bromo-4-{3-[1-(4-bromo-2,5-dichloro-thiophene-3-sulfonyl)-piperidin-4-yl]-propyl}-3,4,5,6-tetrahydro-2H-[1,2′]bipyridinyl-5′-sulfonylamino)-3-phenyl-propionic acid

48. 4-Benzo[b]thiophen-3-yl-3-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-butyric acid

49. 2-(3′-Bromo-4-{3-[1-(4-bromo-5-chloro-thiophene-2-sulfonyl)-piperidin-3-yl]-propoxy}-3,4,5,6-tetrahydro-2H-[1,2′]bipyridinyl-5′-sulfonylamino)-3-(4-chlorophenyl)-propionic acid

50. 3-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-phenyl-propionic acid

51. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(4-methoxyphenyl)-propionic acid

52. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-1,2,3,4-tetrahydronaphthalene-2-carboxylic acid

53. 5-Allyloxycarbonylamino-2-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-pentanoic acid

54. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(4-cyanophenyl)-propionic acid

55. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-indan-2-carboxylic acid

56. 3-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-2-phenyl-propionic acid

57. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(3,5-difluorophenyl)-propionic acid

58. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(2-bromophenyl)-propionic acid

59. 3-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-(4-chlorophenyl)-butyric acid

60. 3-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-(4-chlorophenyl)-butyric acid

61. 2-(3′-Bromo-4-{4-[1-(4-bromo-5-chloro-thiophene-2-sulfonyl)-piperidin-4-yl]-butyl}-3,4,5,6-tetrahydro-2H-[1,2′]bipyridinyl-5′-sulfonylamino)-3-furan-2-yl-propionic acid

62. 1-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-cyclopentanecarboxylic acid

63. 6-Benzyloxycarbonylamino-2-[4-(4-{3-[1-(4-bromo-5-chloro-thiophene-2-sulfonyl)-piperidin-4-yl]-propyl}-piperidin-1-yl)-3-chloro-benzenesulfonylamino]-hexanoic acid

64. [(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-phenethyl-amino]-acetic acid

65. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-(3,4,5-trifluoro-phenyl)-propionic acid

66. 2-[(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-methyl-amino]-3-phenylpropionic acid

67. 3-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-(4-trifluoromethyl-phenyl)-butyric acid

68. 3-[4-(4-{5-[1-(4-Bromo-5-chloro-thiophene-2-sulfonyl)-piperidin-4-yl]-pentyl}-piperidin-1-yl)-3-chloro-benzenesulfonylamino]-cyclopentanecarboxylic acid

69. 4-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-tetrahydrothiopyran-4-carboxylic acid

70. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-hexanoic acid

71. 2-(3′-Bromo-4-{3-[1-(3-bromo-5-chloro-thiophene-2-sulfonyl)-piperidin-4-yl]-propyl}-3,4,5,6-tetrahydro-2H-[1,2′]bipyridinyl-5′-sulfonylamino)-3-phenyl-propionic acid

72. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(3,4-dichlorophenyl)-propionic acid

73. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-methyl-pent-4-enoic acid

74. 2-(3′-Bromo-4-{4-[1-(4-bromo-5-chloro-thiophene-2-sulfonyl)-piperidin-4-yl]-butyl}-3,4,5,6-tetrahydro-2H-[1,2′]bipyridinyl-5′-sulfonylamino)-4-methylsulfanyl-butyric acid

75. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-thiophen-2-yl-propionic acid

76. 2-(3′-Bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonylamino)-4-methanesulfonylbutyric acid

77. 2-(4-{6-[1-(4-Bromo-5-chloro-thiophene-2-sulfonyl)-piperidin-4-yl]-hexyl}-3′-chloro-3,4,5,6-tetrahydro-2H-[1,2′]bipyridinyl-5′-sulfonylamino)-3-phenyl-propionic acid

78. [(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-(2-methoxy-ethyl)-amino]-acetic acid

79. 6-Benzyloxycarbonylamino-2-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-hexanoic acid

80. 2-(4-{3-[1-(4-Bromo-5-chloro-thiophene-2-sulfonyl)-piperidin-4-yl]-propyl}-3′-chloro-3,4,5,6-tetrahydro-2H-[1,2′]bipyridinyl-5′-sulfonylamino)-3-phenyl-propionic acid

81. 2-(4-{3-[1-(4-Bromo-5-chloro-thiophene-2-sulfonyl)-piperidin-4-yl]-propyl}-3′-trifluoromethyl-3,4,5,6-tetrahydro-2H-[1,2′]bipyridinyl-5′-sulfonylamino)-3-phenylpropionic acid

82. 2-(4-{6-[1-(4-Bromo-5-chloro-thiophene-2-sulfonyl)-piperidin-4-yl]-hexyl}-3′-trifluoromethyl-3,4,5,6-tetrahydro-2H-[1,2′]bipyridinyl-5′-sulfonylamino)-3-phenylpropionic acid

83. 3-[4-(4-{7-[1-(4-Bromo-5-chloro-thiophene-2-sulfonyl)-piperidin-4-yl]-heptyl}-piperidin-1-yl)-3-chloro-benzenesulfonylamino]-cyclopentanecarboxylic acid

84. 3-Benzyloxycarbonylamino-2-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-propionic acid

85. 3-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-(4-fluorophenyl)-butyric acid

86. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(3-trifluoromethyl-phenyl)-propionic acid

87. [(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-cyclopentyl-amino]-acetic acid

88. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(2-chlorophenyl)-propionic acid

89. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-cyclohexylpropionic acid

90. 4-[(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-methyl]-benzoic acid

91. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-cyclopropylpropionic acid

92. 2-(3′-Bromo-4-{3-[1-(4-bromo-5-chloro-thiophene-2-sulfonyl)-piperidin-4-yl]-propyl}-3,4,5,6-tetrahydro-2H-[I,2′]bipyridinyl-5′-sulfonylamino)-3-(3,4-difluorophenyl)-propionic acid

93. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-thiazol-4-yl-propionic acid

94. 3-[4-(4-{6-[1-(4-Bromo-5-chloro-thiophene-2-sulfonyl)-piperidin-4-yl]-hexyl}-piperidin-1-yl)-3-chloro-benzenesulfonylamino]-cyclopentanecarboxylic acid

95. [(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-(tetrahydro-furan-2-ylmethyl)-amino]-acetic acid

96. 2-(3′-Bromo-4-{2-[1-(4-bromo-5-chloro-thiophene-2-sulfonyl)-piperidin-4-yl]-ethoxy}-3,4,5,6-tetrahydro-2H-[1,2′]bipyridinyl-5′-sulfonylamino)-3-(4-chlorophenyl)-propionic acid

97. 1-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-cyclohexanecarboxylic acid

98. 2-(3′-Bromo-4-{7-[1-(4-bromo-5-chloro-thiophene-2-sulfonyl)-piperidin-4-yl]-heptyl}-3,4,5,6-tetrahydro-2H-[1,2′]bipyridinyl-5′-sulfonylamino)-3-phenyl-propionic acid

99. 6-Allyloxycarbonylamino-2-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-hexanoic acid

100. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-6-(2,2,2-trifluoroacetylamino)-hexanoic acid

101. 3-[3-Chloro-4-(4-{5-[1-(3,4-dichloro-benzenesulfonyl)-piperidin-4-yl]-pentyl}-piperidin-1-yl)-benzenesulfonylamino]-cyclopentanecarboxylic acid

102. 3-(4-Amino-phenyl)-2-(3′-bromo-4-{3-[1-(4-bromo-5-chloro-thiophene-2-sulfonyl)piperidin-4-yl]-propyl}-3,4,5,6-tetrahydro-2H-[1,2′]bipyridinyl-5′-sulfonylamino)-propionic acid

103. 2-(3′-Bromo-4-{7-[1-(4-bromo-5-chloro-thiophene-2-sulfonyl)-piperidin-4-yl]-heptyl}-3,4,5,6-tetrahydro-2H-[1,2′]bipyridinyl-5′-sulfonylamino)-4-methylsulfanylbutyric acid

104. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(4-tert-butylphenyl)-propionic acid

105. 3-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-5-methylsulfanylpentanoic acid

106. 3-[4-(4-{3-[1-(4-Bromo-5-chloro-thiophene-2-sulfonyl)-piperidin-4-yl]-propyl}-piperidin-1-yl)-3-chloro-benzenesulfonylamino]-cyclohexanecarboxylic acid

107. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(4-fluorophenyl)-propionic acid

108. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(4-iodo-phenyl)propionic acid

109. 2-(4-{4-[1-(4-Bromo-5-chloro-thiophene-2-sulfonyl)-piperidin-4-yl]-butyl}-3′-trifluoromethyl-3,4,5,6-tetrahydro-2H-[1,2′]bipyridinyl-5′-sulfonylamino)-3-phenylpropionic acid

110. [(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-(3-phenyl-propyl)amino]-acetic acid

111. 2-(3′-Bromo-4-{3-[1-(4-bromo-5-chloro-thiophene-2-sulfonyl)-piperidin-4-yl]-propoxy}-3,4,5,6-tetrahydro-2H-[1,2′]bipyridinyl-5′-sulfonylamino)-3-(4-chlorophenyl)-propionic acid

112. 2-(3′-Bromo-4-{4-[1-(4-iodo-benzenesulfonyl)-piperidin-4-yl]-butyl}-3,4,5,6-tetrahydro-2H-[1,2′]bipyridinyl-5′-sulfonylamino)-3-phenyl-propionic acid

113. 1-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-cyclopropanecarboxylic acid

114. 3-[4-(4-{4-[1-(4-Bromo-5-chloro-thiophene-2-sulfonyl)-piperidin-4-yl]-butyl}-piperidin-1-yl)-3-chloro-benzenesulfonylamino]-cyclohexanecarboxylic acid

115. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-o-tolyl-propionic acid

116. [1-(3′-Bromo-4-{3-[1-(4-bromo-5-chloro-thiophene-2-sulfonyl)-piperidin-4-yl]-propyl}-3,4,5,6-tetrahydro-2H-[1,2]bipyridinyl-5′-sulfonyl)-pyrrolidin-2-yl]-acetic acid

117. 3-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-benzoic acid

118. {(3′-Bromo-4-{3-[1-(4-bromo-2,5-dichloro-thiophene-3-sulfonyl)-piperidin-4-yl]-propyl}-3,4,5,6-tetrahydro-2H-[1,2′]bipyridinyl-5′-sulfonyl)-[2-(4-chloro-phenyl)-ethyl]-amino}-acetic acid

119. 3-[4-(4-{3-[1-(5-Bromo-6-chloro-pyridine-3-sulfonyl)-piperidin-4-yl]-propyl}-piperidin-1-yl)-3-chloro-benzenesulfonylamino]-cyclopentanecarboxylic acid

120. 6-Allyloxycarbonylamino-2-(3′-bromo-4-{3-[1-(3-bromo-5-chloro-thiophene-2-sulfonyl)-piperidin-4-yl]-propyl}-3,4,5,6-tetrahydro-2H-[1,2′]bipyridinyl-5′-sulfonylamino)-hexanoic acid

121. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(4-trifluoromethyl-phenyl)-propionic acid

122. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-(4-methoxy-phenyl)-propionic acid

123. 3-[4-(4-{5-[1-(Benzo[b]thiophene-2-sulfonyl)-piperidin-4-yl]-pentyl}-piperidin-1-yl)-3-chloro-benzenesulfonylamino]-cyclopentanecarboxylic acid

124. {[4-(4-{3-[1-(4-Bromo-5-chloro-thiophene-2-sulfonyl)-piperidin-4-yl]-propyl}-piperidin-1-yl)-3-chloro-benzenesulfonyl]-methyl-amino}-acetic acid

125. 3-[4-(4-{3-[1-(4-Bromo-5-chloro-thiophene-2-sulfonyl)-piperidin-4-yl]-propyl}-piperidin-1-yl)-3-chloro-benzenesulfonylamino]-cyclopentanecarboxylic acid

126. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-6-[(pyridine-3-carbonyl)-amino]-hexanoic acid

127. 3-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-phenyl-butyric acid

128. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-methylsulfanylbutyric acid

129. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-6-[(furan-2-carbonyl)-amino]-hexanoic acid

130. 3-[4-(4-{2-[1-(4-Bromo-5-chloro-thiophene-2-sulfonyl)-piperidin-4-yl]-ethyl}-piperidin-1-yl)-3-chloro-benzenesulfonylamino]-cyclopentanecarboxylic acid

131. 3-[4-(4-{3-[1-(4-Bromo-5-chloro-thiophene-2-sulfonyl)-piperidin-4-yl]-propyl}-piperidin-1-yl)-3-chloro-benzenesulfonylamino]-cyclopentanecarboxylic acid

132. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-naphthalen-1-yl-propionic acid

133. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-4-methyl-pentanoic acid

134. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(3H-indol-3-yl)-propionic acid

135. 3-[4-(4-{3-[1-(4-Bromo-5-chloro-thiophene-2-sulfonyl)-piperidin-4-yl]-propyl}-piperidin-1-yl)-3-chloro-benzenesulfonylamino]-3-phenyl-propionic acid

136. 3-[4-(4-{5-[1-(Benzo[b]thiophene-2-sulfonyl)-piperidin-4-yl]-pentyl}-piperidin-1-yl)-3-chloro-benzenesulfonylamino]-cyclohexanecarboxylic acid

137. 2-(3′-Bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonylamino)-3-(4-bromophenyl)-propionic acid

138. 3-[4-(4-{3-[1-(4-Bromo-5-chloro-thiophene-2-sulfonyl)-piperidin-4-yl]-propyl}-piperidin-1-yl)-3-chloro-benzenesulfonylamino]-cyclopentanecarboxylic acid

139. 2-(4-{4-[1-(4-Bromo-5-chloro-thiophene-2-sulfonyl)-piperidin-4-yl]-butyl}-3′-chloro-3,4,5,6-tetrahydro-2H-[1,2′]bipyridinyl-5′-sulfonylamino)-3-phenyl-propionic acid

140. 1-[4-(4-{3-[1-(4-Bromo-5-chloro-thiophene-2-sulfonyl)-piperidin-4-yl]-propyl}-piperidin-1-yl)-3-chloro-benzenesulfonylamino]-3-hydroxy-cyclopentanecarboxylic acid

141. {(3′-Bromo-4-{3-[1-(3-bromo-5-chloro-thiophene-2-sulfonyl)-piperidin-4-yl]-propyl}-3,4,5,6-tetrahydro-2H-[1,2′]bipyridinyl-5′-sulfonyl)-[2-(4-chloro-phenyl)-ethyl]-amino}-acetic acid

142. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-(4-trifluoromethyl-phenyl)-propionic acid

143. 3-[3-Chloro-4-(4-{3-[1-(4,5-dichloro-thiophene-2-sulfonyl)-piperidin-4-yl]-propyl}-piperidin-1-yl)-benzenesulfonylamino]-cyclopentanecarboxylic acid

144. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-6-(cyclopropanecarbonyl-amino)-hexanoic acid

145. 3-[4-(4-{3-[1-(4-Bromo-5-chloro-thiophene-2-sulfonyl)-piperidin-4-yl]-propyl}-piperidin-1-yl)-3-chloro-benzenesulfonylamino]-cyclopentanecarboxylic acid

146. 3-[4-(4-{3-[1-(4-Bromo-5-chloro-thiophene-2-sulfonyl)-piperidin-4-yl]-propyl}-piperidin-1-yl)-3-chloro-benzenesulfonylamino]-3-phenyl-propionic acid

147. 3-[4-(4-{3-[1-(5-Bromo-6-chloro-pyridine-3-sulfonyl)-piperidin-4-yl]-propyl}-piperidin-1-yl)-3-chloro-benzenesulfonylamino]-cyclohexanecarboxylic acid

148. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-pyridin-4-yl-propionic acid

149. 2-(3′-Bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonylamino)-3-(4-chlorophenyl)-propionic acid

150. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-cyclohexyl-propionic acid

151. 3-[3-Chloro-4-(4-{5-[1-(3,4-dichloro-benzenesulfonyl)-piperidin-4-yl]-pentyl}-piperidin-1-yl)-benzenesulfonylamino]-cyclohexanecarboxylic acid

152. 3-[4-(4-{5-[1-(4-Bromo-5-chloro-thiophene-2-sulfonyl)-piperidin-4-yl]-pentyl}-piperidin-1-yl)-3-chloro-benzenesulfonylamino]-cyclohexanecarboxylic acid

153. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-phenyl-propionic acid

154. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-phenyl-propionic acid

155. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-4-methylsulfanyl-butyric acid

156. 4-[4-(4-{3-[1-(4-Bromo-5-chloro-thiophene-2-sulfonyl)-piperidin-4-yl]-propyl}-piperidin-1-yl)-3-chloro-benzenesulfonyl]-morpholine-2-carboxylic acid

157. 3-Allyloxycarbonylamino-2-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-propionic acid

158. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-pent-4-ynoic acid

159. 1-[4-(4-{3-[1-(4-Bromo-5-chloro-thiophene-2-sulfonyl)-piperidin-4-yl]-propyl}-piperidin-1-yl)-3-chloro-benzenesulfonyl]-piperidine-3-carboxylic acid

160. 2-[3-Chloro-4-(4-{3-[1-(3,4-dichloro-benzenesulfonyl)-piperidin-4-yl]-propyl}-piperidin-1-yl)-benzenesulfonylamino]-3-phenyl-propionic acid

161. 1-[4-(4-{3-[1-(4-Bromo-5-chloro-thiophene-2-sulfonyl)-piperidin-4-yl]-propyl}-piperidin-1-yl)-3-chloro-benzenesulfonyl]-piperidine-2-carboxylic acid

162. 3-[4-(4-{5-[1-(Benzofuran-2-sulfonyl)-piperidin-4-yl]-pentyl}-piperidin-1-yl)-3-chloro-benzenesulfonylamino]-cyclohexanecarboxylic acid

163. 3-[3-Chloro-4-(4-{3-[1-(4,5-dibromo-thiophene-2-sulfonyl)-piperidin-4-yl]-propyl}-piperidin-1-yl)-benzenesulfonylamino]-cyclopentanecarboxylic acid

164. 3-[4-(4-{3-[1-(4-Bromo-5-chloro-thiophene-2-sulfonyl)-piperidin-4-yl]-propyl}-piperidin-1-yl)-3-chloro-benzenesulfonylamino]-cyclopentanecarboxylic acid

165. 4-Allyloxycarbonylamino-2-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-butyric acid

166. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-propionic acid

167. 4-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-tetrahydro-pyran-4-carboxylic acid

168. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-cyclohexylpropionic acid

169. 2-[3′-Bromo-4-(2-{2-[1-(4-bromo-5-chloro-thiophene-2-sulfonyl)-piperidin-4-yl]-ethoxy}-ethyl)-3,4,5,6-tetrahydro-2H-[1,2′]bipyridinyl-5′-sulfonylamino]-3-phenylpropionic acid

170. {(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-[2-(4-chloro-phenyl)-ethyl]-amino}-acetic acid

171. 3-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-pyridin-4-yl-butyric acid

172. 2-(4-{4-[1-(4-Bromo-5-chloro-thiophene-2-sulfonyl)-piperidin-4-yl]-butyl}-3,4,5,6-tetrahydro-2H-[1,2′]bipyridinyl-5′-sulfonylamino)-3-phenyl-propionic acid

173. 1-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-hydroxycyclopentanecarboxylic acid

174. 1-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-hydroxycyclohexanecarboxylic acid

175. 3-[4-(4-{3-[1-(5-Bromo-6-chloro-pyridine-3-sulfonyl)-piperidin-4-yl]-propyl}-piperidin-1-yl)-3-chloro-benzenesulfonylamino]-propionic acid

176. 3-[4-(4-{3-[1-(5-Bromo-6-chloro-pyridine-3-sulfonyl)-piperidin-4-yl]-propyl}-piperidin-1-yl)-3-chloro-benzenesulfonylamino]-cyclopentanecarboxylic acid

177. 3-[4-(4-{4-[1-(4-Bromo-5-chloro-thiophene-2-sulfonyl)-piperidin-4-yl]-butyl}-piperidin-1-yl)-3-chloro-benzenesulfonylamino]-cyclopentanecarboxylic acid

178. 3-[3-Chloro-4-(4-{3-[1-(4,5-dichloro-thiophene-2-sulfonyl)-piperidin-4-yl]-propyl}-piperidin-1-yl)-benzenesulfonylamino]-cyclohexanecarboxylic acid

179. 3-[3-Chloro-4-(4-{3-[1-(3,4-dichloro-benzenesulfonyl)-piperidin-4-yl]-propyl}-piperidin-1-yl)-benzenesulfonylamino]-cyclopentanecarboxylic acid

180. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-methoxy-butyric acid

181. 2-(3′-Bromo-4-{3-[1-(4-bromo-5-chloro-thiophene-2-sulfonyl)-piperidin-3-yloxy]-propyl}-3,4,5,6-tetrahydro-2H-[1,2′]bipyridinyl-5′-sulfonylamino)-3-(4-chlorophenyl)-propionic acid

182. 3-[3-Chloro-4-(4-{3-[1-(4,5-dichloro-thiophene-2-sulfonyl)-piperidin-4-yl]-propyl}-piperidin-1-yl)-benzenesulfonylamino]-cyclopentanecarboxylic acid

183. 3-[3-Chloro-4-(4-{3-[1-(4,5-dibromo-thiophene-2-sulfonyl)-piperidin-4-yl]-propyl}-piperidin-1-yl)-benzenesulfonylamino]-cyclopentanecarboxylic acid

184. 1-[4-(4-{3-[1-(4-Bromo-5-chloro-thiophene-2-sulfonyl)-piperidin-4-yl]-propyl}-piperidin-1-yl)-3-chloro-benzenesulfonyl]-pyrrolidine-2-carboxylic acid

185. 3-[4-(4-{5-[1-(4-Bromo-5-chloro-thiophene-2-sulfonyl)-piperidin-4-yl]-pentyl}-piperidin-1-yl)-3-chloro-benzenesulfonylamino]-cyclopentanecarboxylic acid

186. 3-[4-(4-{3-[1-(4-Bromo-5-chloro-thiophene-2-sulfonyl)-piperidin-4-yl]-propyl}-piperidin-1-yl)-3-chloro-benzenesulfonylamino]-propionic acid

187. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-6-(3-ethyl-ureido)-hexanoic acid

188. 3-[3-Chloro-4-(4-{3-[1-(3,4-dichloro-benzenesulfonyl)-piperidin-4-yl]-propyl}-piperidin-1-yl)-benzenesulfonylamino]-cyclopentanecarboxylic acid

189. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(4-hydroxyphenyl)-propionic acid

190. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-methanesulfonyl-butyric acid

191. 2-(4-{5-[1-(6-Chloro-5-trifluoromethyl-pyridine-3-sulfonyl)-piperidin-4-yl]-pentyl}-3′-trifluoromethyl-3,4,5,6-tetrahydro-2H-[1,2′]bipyridinyl-5′-sulfonylamino)-3-phenyl-propionic acid

192. 2-(4-{5-[1-(3-Fluoro-4-trifluoromethyl-benzenesulfonyl)-piperidin-4-yl]-pentyl}-3′-trifluoromethyl-3,4,5,6-tetrahydro-2H-[1,2′]bipyridinyl-5′-sulfonylamino)-3-phenylpropionic acid

193. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-(1 H-indol-3-yl)-propionic acid

194. 2-(3′-Bromo-4-{3-[1-(4-tert-butyl-benzenesulfonyl)-piperidin-4-yl]-propyl}-3,4,5,6-tetrahydro-2H-[1,2′]bipyridinyl-5′-sulfonylamino)-3-phenyl-propionic acid

195. 3-[4-(4-{3-[1-(4-Bromo-5-chloro-thiophene-2-sulfonyl)-piperidin-4-yl]-propyl}-piperidin-1-yl)-3-chloro-benzenesulfonylamino]-cyclopentanecarboxylic acid

196. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-methylpentanoic acid

197. 3-[4-(4-{3-[1-(4-Bromo-5-chloro-thiophene-2-sulfonyl)-piperidin-4-yl]-propyl}-piperidin-1-yl)-3-chloro-benzenesulfonylamino]-3-pyridin-3-yl-propionic acid

198. 1-[4-(4-{3-[1-(4-Bromo-5-chloro-thiophene-2-sulfonyl)-piperidin-4-yl]-propyl}-piperidin-1-yl)-3-chloro-benzenesulfonyl]-azetidine-3-carboxylic acid

199. 3-[3-Chloro-4-(4-{3-[1-(4-trifluoromethyl-benzenesulfonyl)-piperidin-4-yl]-propyl}-piperidin-1-yl)-benzenesulfonylamino]-cyclopentanecarboxylic acid

200. 3-[4-(4-{2-[1-(4-Bromo-5-chloro-thiophene-2-sulfonyl)-piperidin-4-yl]-ethyl}-piperidin-1-yl)-3-chloro-benzenesulfonylamino]-cyclopentanecarboxylic acid

201. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-propionic acid

202. 6-Allyloxycarbonylamino-2-(3′-bromo-4-{4-[1-(4-bromo-5-chloro-thiophene-2-sulfonyl)-piperidin-4-yl]-butyl}-3,4,5,6-tetrahydro-2H-[1,2′]bipyridinyl-5′-sulfonylamino)-hexanoic acid

203. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-2-methyl-propionic acid

204. 2-(3′-Bromo-4-{3-[1-(4-tert-butyl-benzenesulfonyl)-piperidin-4-yl]-propyl}-3,4,5,6-tetrahydro-2H-[1,2′]bipyridinyl-5′-sulfonylamino)-propionic acid

205. 2-[3-Chloro-4-(4-{3-[1-(3,4-dichloro-benzenesulfonyl)-piperidin-4-yl]-propyl}-piperidin-1-yl)-benzenesulfonylamino]-4-methylsulfanyl-butyric acid

206. 2-[4-(4-{3-[1-(4-tert-Butyl-benzenesulfonyl)-piperidin-4-yl]-propyl}-piperidin-1-yl)-3-chloro-benzenesulfonylamino]-3-phenyl-propionic acid

207. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-6-propionylaminohexanoic acid

208. 2-[3-Chloro-4-(4-{3-[1-(3,4-dichloro-benzenesulfonyl)-piperidin-4-yl]-propyl}-piperidin-1-yl)-benzoylamino]-4-methylsulfanyl-butyric acid

209. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-methyl-butyric acid

210. 2-[3-Chloro-4-(4-{3-[1-(3,4-dichloro-benzenesulfonyl)-piperidin-4-yl]-propyl}-piperidin-1-yl)-benzoylamino]-3-phenyl-propionic acid

211. 2-(3′-Bromo-4-{3-[1-(4-trifluoromethyl-benzenesulfonyl)-piperidin-4-yl]-propyl}-3,4,5,6-tetrahydro-2H-[1,2′]bipyridinyl-5′-sulfonylamino)-3-phenyl-propionic acid

212. (3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-indan-2-yl-acetic acid

213. 3-[3-Chloro-4-(4-{3-[1-(4,5-dichloro-thiophene-2-sulfonyl)-piperidin-4-yl]-propyl}-piperidin-1-yl)-benzenesulfonylamino]-cyclopentanecarboxylic acid

214. 2-(3′-Bromo-4-{3-[1-(4-trifluoromethyl-benzenesulfonyl)-piperidin-4-yl]-propyl}-3,4,5,6-tetrahydro-2H-[1,2′]bipyridinyl-5′-sulfonylamino)-4-methylsulfanyl-butyric acid

215. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-phenyl-butyric acid

216. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-carbamoylbutyric acid

217. 3-[4-(4-{3-[1-(4-Bromo-5-chloro-thiophene-2-sulfonyl)-piperidin-4-yl]-propyl}-piperidin-1-yl)-3-chloro-benzenesulfonylamino]-cyclopentanecarboxylic acid

218. 3-[3-Chloro-4-(4-{4-[1-(3,4-dichloro-benzenesulfonyl)-piperidin-4-yl]-butyl}-piperidin-1-yl)-benzenesulfonylamino]-cyclohexanecarboxylic acid

219. 3-[3-Chloro-4-(4-{3-[1-(4-trifluoromethyl-benzenesulfonyl)-piperidin-4-yl]-propyl}-piperidin-1-yl)-benzenesulfonylamino]-cyclopentanecarboxylic acid

220. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-hexanoic acid

221. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-hydroxy-butyric acid

222. 6-Acetylamino-2-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-hexanoic acid

223. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-phenyl-propionic acid

224. 1-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-4-hydroxy-pyrrolidine-2-carboxylic acid

225. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-methanesulfonylbutyric acid

226. 2-[5-Bromo-6-( {8-[(4-bromo-5-chloro-thiophene-2-sulfonyl)-methyl-amino]-octyl}-methyl-amino)-pyridine-3-sulfonylamino]-3-(4-chloro-phenyl)-propionic acid

227. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-phenyl-propionic acid

228. 2-[4-(4-{3-[1-(4-tert-Butyl-benzenesulfonyl)-piperidin-4-yl]-propyl}-piperidin-1-yl)-3-chloro-benzenesulfonylamino]-4-methylsulfanyl-butyric acid

229. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(3H-indol-3-yl)-propionic acid

230. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-hydroxy-butyric acid

231. 1-[4-(4-{3-[1-(5-Bromo-6-methoxy-pyridine-3-sulfonyl)-piperidin-4-yl]-propyl}-piperidin-1-yl)-3-chloro-benzenesulfonyl]-4-hydroxy-pyrrolidine-2-carboxylic acid

232. 2-[4-(4-{3-[1-(4-tert-Butyl-benzenesulfonyl)-piperidin-4-yl]-propyl}-piperidin-1-yl)-3-chloro-benzenesulfonylamino]-3-(4-hydroxy-phenyl)-propionic acid

233. 3-[3-Chloro-4-(4-{3-[1-(5-chloro-benzo [1,2,5]oxadiazole-4-sulfonyl)-piperidin-4-yl]-propyl}-piperidin-1-yl)-benzenesulfonylamino]-cyclopentanecarboxylic acid

234. 6-Allyloxycarbonylamino-2-(3′-bromo-4-{3-[1-(4-bromo-2,5-dichloro-thiophene-3-sulfonyl)-piperidin-4-yl]-propyl}-3,4,5,6-tetrahydro-2H-[1,2′]bipyridinyl-5′-sulfonylamino)-hexanoic acid

235. 2-(3′-Bromo-4-{3-[1-(4-trifluoromethyl-benzenesulfonyl)-piperidin-4-yl]-propyl}-3,4,5,6-tetrahydro-2H-[1,2′]bipyridinyl-5′-sulfonylamino)-3-hydroxy-propionic acid

236. 2-(3′-Bromo-4-{3-[1-(4-tert-butyl-benzenesulfonyl)-piperidin-4-yl]-propyl}-3,4,5,6-tetrahydro-2H-[1,2′]bipyridinyl-5′-sulfonylamino)-3-(4-hydroxy-phenyl)-propionic acid

237. 2-[4-(4-{3-[1-(4-tert-Butyl-benzenesulfonyl)-piperidin-4-yl]-propyl}-piperidin-1-yl)-3-chloro-benzenesulfonylamino]-propionic acid

238. 2-[(4-{3-[1-(3,4-Dichloro-benzenesulfonyl)-piperidin-4-yl]-propyl}-3,4,5,6-tetrahydro-2H-[1,2′]bipyridinyl-5′-carbonyl)-amino]-3-phenyl-propionic acid

239. 2-[4-(4-{3-[1-(4-Bromo-5-chloro-thiophene-2-sulfonyl)-piperidin-4-yl]-propyl}-piperidin-1-yl)-3-chloro-benzoylamino]-3-phenyl-propionic acid

240. 2-[4-(4-{3-[1-(4-tert-Butyl-benzenesulfonyl)-piperidin-4-yl]-propyl}-piperidin-1-yl)-3-chloro-benzenesulfonylamino]-3-methyl-butyric acid

241. 3-Cyclopenta-1,3-dienyl-2-(4-{3-[1-(3,4-dichloro-benzenesulfonyl)-piperidin-4-yl]-propyl}-3,4,5,6-tetrahydro-2H-[1,2′]bipyridinyl-5′-sulfonylamino)-propionic acid

242. 2-[4-(4-{3-[1-(4-tert-Butyl-benzenesulfonyl)-piperidin-4-yl]-propyl}-piperidin-1-yl)-3-chloro-benzenesulfonylamino]-3-hydroxy-propionic acid

243. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-hydroxypropionic acid

244. 2-(3′-Bromo-4-{3-[1-(4-tert-butyl-benzenesulfonyl)-piperidin-4-yl]-propyl}-3,4,5,6-tetrahydro-2H-[1,2′]bipyridinyl-5′-sulfonylamino)-3-hydroxy-propionic acid

245. 3-[3-Chloro-4-(4-{4-[1-(2-chloro-[4-(3-carboxy)cyclopentylsulfamoyl]-phenyl)-piperidin-4-yl]-butyl}-piperidin-1-yl)-benzenesulfonylamino]-cyclopentanecarboxylic acid

246. [4-(4-{3-[1-(4-Bromo-5-chloro-thiophene-2-sulfonyl)-piperidin-4-yl]-propyl}-piperidin-1-yl)-3-chloro-benzenesulfonyl]-acetic acid

247. 2-(3′-Bromo-4-{4-[1-(4-bromo-5-chloro-thiophene-2-sulfonyl)-piperidin-4-yl]-butyl}-3,4,5,6-tetrahydro-2H-[1,2′]bipyridinyl-5′-sulfonylamino)-3-(4-fluoro-phenyl)-propionic acid

248. 2-[3-Chloro-4-(4-{3-[1-(3,4-dichloro-benzoyl)-piperidin-4-yl]-propyl}-piperidin-1-yl)-benzoylamino]-4-methylsulfanyl-butyric acid

249. 2-[4-(4-{3-[1-(4-tert-Butyl-benzenesulfonyl)-piperidin-4-yl]-propyl}-piperidin-1-yl)-3-chloro-benzenesulfonylamino]-4-methyl-pentanoic acid

250. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-(4-hydroxy-phenyl)-propionic acid

251. 3-[3-Chloro-4-(4-{3-[1-(4-chloro-benzenesulfonyl)-piperidin-4-yl]-propyl}-piperidin-1-yl)-benzenesulfonylamino]-cyclopentanecarboxylic acid

252. 3-[3-Chloro-4-(4-{3-[1-(4-chloro-benzenesulfonyl)-piperidin-4-yl]-propyl}-piperidin-1-yl)-benzenesulfonylamino]-cyclopentanecarboxylic acid

253. 1-[4-(4-{3-[1-(4-Bromo-5-chloro-thiophene-2-sulfonyl)-piperidin-4-yl]-propyl}-piperidin-1-yl)-3-chloro-benzenesulfonyl]-4-hydroxy-pyrrolidine-2-carboxylic acid

254. 2-(3′-Bromo-4-{3-[1-(4-tert-butyl-benzenesulfonyl)-piperidin-4-yl]-propyl}-3,4,5,6-tetrahydro-2H-[1,2′]bipyridinyl-5′-sulfonylamino)-3-methyl-butyric acid

255. 6-Allyloxycarbonylamino-2-(3′-bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonylamino)-hexanoic acid

256. 3-[3-Chloro-4-(4-{3-[1-(5-chloro-thiophene-2-sulfonyl)-piperidin-4-yl]-propyl}-piperidin-1-yl)-benzenesulfonylamino]-cyclopentanecarboxylic acid

257. 2-[3-Chloro-4-(4-{3-[1-(3,4-dichloro-benzoyl)-piperidin-4-yl]-propyl}-piperidin-1-yl)-benzoylamino]-3-phenyl-propionic acid

258. 3-[3-Chloro-4-(4-{4-[1-(3,4-dichloro-benzenesulfonyl)-piperidin-4-yl]-butyl}-piperidin-1-yl)-benzenesulfonylamino]-cyclopentanecarboxylic acid

259. 3-[3-Chloro-4-(4-{3-[1-(3-chloro-benzenesulfonyl)-piperidin-4-yl]-propyl}-piperidin-1-yl)-benzenesulfonylamino]-cyclopentanecarboxylic acid

260. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-hydroxy-propionic acid

261. 2-(4-{3-[1-(3,4-Dichloro-benzenesulfonyl)-piperidin-4-yl]-propyl}-3,4,5,6-tetrahydro-2H-[1,2′]bipyridinyl-5′-sulfonylamino)-4-methylsulfanyl-butyric acid

262. 2-(3′-Bromo-4-{3-[1-(4-trifluoromethyl-benzenesulfonyl)-piperidin-4-yl]-propyl}-3,4,5,6-tetrahydro-2H-[1,2′]bipyridinyl-5′-sulfonylamino)-3-(4-hydroxy-phenyl)-propionic acid

263. 3-[3-Chloro-4-(4-{3-[1-(toluene-4-sulfonyl)-piperidin-4-yl]-propyl}-piperidin-1-yl)-benzenesulfonylamino]-cyclopentanecarboxylic acid

264. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-3-methyl-butyric acid

265. 3-[3-Chloro-4-(4-{3-[1-(3-chloro-benzenesulfonyl)-piperidin-4-yl]-propyl}-piperidin-1-yl)-benzenesulfonylamino]-cyclopentanecarboxylic acid

266. 2-(3′-Bromo-4-{3-[1-(4-tert-butyl-benzenesulfonyl)-piperidin-4-yl]-propyl}-3,4,5,6-tetrahydro-2H-[1,2′]bipyridinyl-5′-sulfonylamino)-succinic acid

267. 2-[(4-{3-[1-(3,4-Dichloro-benzenesulfonyl)-piperidin-4-yl]-propyl}-3,4,5,6-tetrahydro-2H-[1,2′]bipyridinyl-5′-carbonyl)-amino]-4-methylsulfanyl-butyric acid

268. 3-[3-Chloro-4-(4-{3-[1-(5-chloro-benzo[1,2,5]oxadiazole-4-sulfonyl)-piperidin-4-yl]-propyl}-piperidin-1-yl)-benzenesulfonylamino]-cyclopentanecarboxylic acid

269. 3-Allyloxycarbonylamino-2-(3′-bromo-4-{3-[1-(3-bromo-5-chloro-thiophene-2-sulfonyl)-piperidin-4-yl]-propyl}-3,4,5,6-tetrahydro-2H-[1,2′]bipyridinyl-5′-sulfonylamino)-propionic acid

270. 3-[3-Chloro-4-(4-{3-[1-(5-chloro-thiophene-2-sulfonyl)-piperidin-4-yl]-propyl}-piperidin-1-yl)-benzenesulfonylamino]-cyclopentanecarboxylic acid

271. 2-{5-Bromo-6-[9-(4-bromo-5-chloro-thiophene-2-sulfonylamino)-nonylamino]-pyridine-3-sulfonylamino}-3-(4-chloro-phenyl)-propionic acid

272. 3-[3-Chloro-4-(4-{3-[1-(toluene-4-sulfonyl)-piperidin-4-yl]-propyl}-piperidin-1-yl)-benzenesulfonylamino]-cyclopentanecarboxylic acid

273. 3-[3-Chloro-4-(4-{3-[1-(4-fluoro-benzenesulfonyl)-piperidin-4-yl]-propyl}-piperidin-1-yl)-benzenesulfonylamino]-cyclopentanecarboxylic acid

274. 2-[(4-{3-[1-(3,4-Dichloro-benzoyl)-piperidin-4-yl]-propyl}-3,4,5,6-tetrahydro-2H-[1,2′]bipyridinyl-5′-carbonyl)-amino]-3-phenyl-propionic acid

275. 2-(3′-Bromo-4-{3-[1-(4-tert-butyl-benzenesulfonyl)-piperidin-4-yl]-propyl}-3,4,5,6-tetrahydro-2H-[1,2′]bipyridinyl-5′-sulfonylamino)-4-methyl-pentanoic acid

276. 2-[3-Chloro-4-(4-{3-[1-(3,4-dichloro-benzoyl)-piperidin-4-yl]-propyl}-piperidin-1-yl)-benzenesulfonylamino]-4-methylsulfanyl-butyric acid

277. 2-(3′-Bromo-4-{3-[1-(4-trifluoromethyl-benzenesulfonyl)-piperidin-4-yl]-propyl}-3,4,5,6-tetrahydro-2H-[I,2′]bipyridinyl-5′-sulfonylamino)-3-methyl-butyric acid

278. 3-[3-Chloro-4-(4-{3-[1-(4-fluoro-benzenesulfonyl)-piperidin-4-yl]-propyl}-piperidin-1-yl)-benzenesulfonylamino]-cyclopentanecarboxylic acid

279. 2-(3′-Bromo-4-{3-[1-(4-tert-butyl-benzenesulfonyl)-piperidin-4-yl]-propyl}-3,4,5,6-tetrahydro-2H-[1,2′]bipyridinyl-5′-sulfonylamino)-4-methylsulfanyl-butyric acid

280. 2-(4-{3-[1-(3,4-Dichloro-benzoyl)-piperidin-4-yl]-propyl}-3,4,5,6-tetrahydro-2H-[1,2′]bipyridinyl-5′-sulfonylamino)-3-phenyl-propionic acid

281. 3-[4-(4-{3-[1-(4-Bromo-5-chloro-thiophene-2-sulfonyl)-piperidin-4-yl]-propyl}-piperidin-1-yl)-3-chloro-benzenesulfonylamino]-cyclopentanecarboxylic acid hydroxyamide

282. 2-(3′-Bromo-4-{3-[1-(4-trifluoromethyl-benzenesulfonyl)-piperidin-4-yl]-propyl}-3,4,5,6-tetrahydro-2H-[1,2′]bipyridinyl-5′-sulfonylamino)-4-methyl-pentanoic acid

283. 3-(4-{4-[3-(1-Benzenesulfonyl-piperidin-4-yl)-propyl]-piperidin-1-yl}-3-chlorobenzenesulfonylamino)-cyclopentanecarboxylic acid

284. 2-(4-{3-[1-(3,4-Dichloro-benzoyl)-piperidin-4-yl]-propyl}-3,4,5,6-tetrahydro-2H-[1,2′]bipyridinyl-5′-sulfonylamino)-4-methylsulfanyl-butyric acid

285. 2-[3-Chloro-4-(4-{3-[1-(3,4-dichloro-benzoyl)-piperidin-4-yl]-propyl}-piperidin-1-yl)-benzenesulfonylamino]-3-phenyl-propionic acid

286. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-succinic acid

287. 3-(4-{4-[3-(1-Benzenesulfonyl-piperidin-4-yl)-propyl]-piperidin-1-yl}-3-chlorobenzenesulfonylamino)-cyclopentanecarboxylic acid

288. 2-(3′-Bromo-4-{4-[1-(4-bromo-5-chloro-thiophene-2-sulfonyl)-piperidin-4-yl]-butyl}-3,4,5,6-tetrahydro-2H-[1,2′]bipyridinyl-5′-sulfonylamino)-3-(4-chloro-phenyl)-propionic acid

289. 3′-Bromo-4-{3-[1-(5-chloro-benzo[1,2,5]oxadiazole-4-sulfonyl)-piperidin-4-yl]-propyl}-5′-([1,4]diazepane-1-sulfonyl)-3,4,5,6-tetrahydro-2H-[1,2′]bipyridinyl

290. 2-[4-(4-{3-[1-(4-Bromo-5-chloro-thiophene-2-sulfonyl)-piperidin-4-yl]-propyl}-piperidin-1-yl)-3-chloro-benzenesulfonylamino]-succinic acid

291. 2-[(4-{3-[1-(3,4-Dichloro-benzoyl)-piperidin-4-yl]-propyl}-3,4,5,6-tetrahydro-2H-[1,2′]bipyridinyl-5′-carbonyl)-amino]-4-methylsulfanyl-butyric acid

292. 3-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-4-hydroxy-butyric acid

293. 3-[4-(4-{4-[1-(Benzo[b]thiophene-2-sulfonyl)-piperidin-4-yl]-butyl}-piperidin-1-yl)-3-chloro-benzenesulfonylamino]-cyclopentanecarboxylic acid

294. 2-[4-(4-{3-[1-(4-tert-Butyl-benzenesulfonyl)-piperidin-4-yl]-propyl}-piperidin-1-yl)-3-chloro-benzenesulfonylamino]-succinic acid

295. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(4-methylaminophenyl)-propionic acid

296. 2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(1 H-imidazol-4-yl)-propionic acid

297. 3-[4-(4-{4-[1-(Benzo[b]thiophene-2-sulfonyl)-piperidin-4-yl]-butyl}-piperidin-1-yl)-3-chloro-benzenesulfonylamino]-cyclohexanecarboxylic acid

298. 2-[3-Chloro-4-(4-{3-[1-(thiazolidine-4-carbonyl)-piperidin-4-yl]-propyl}-piperidin-1-yl)-benzoylamino]-3-phenyl-propionic acid

299. 6-Amino-2-[4-(4-{3-[1-(4-tert-butyl-benzenesulfonyl)-piperidin-4-yl]-propyl}-piperidin-1-yl)-3-chloro-benzenesulfonylamino]-hexanoic acid

300. 1-[4-(4-{3-[1-(4-Bromo-5-chloro-thiophene-2-sulfonyl)-piperidin-4-yl]-propyl}-piperidin-1-yl)-3-chloro-benzenesulfonyl]-piperazine-2-carboxylic acid

301. 4-[4-(4-{3-[1-(4-Bromo-5-chloro-thiophene-²-sulfonyl)-piperidin-4-yl]-propyl}-piperidin-1-yl)-3-chloro-benzenesulfonyl]-piperazine-2-carboxylic acid

302. 6-Amino-2-(3′-bromo-4-{3-[1-(4-tert-butyl-benzenesulfonyl)-piperidin-4-yl]-propyl}-3,4,5,6-tetrahydro-2H-[1,2′]bipyridinyl-5′-sulfonylamino)-hexanoic acid

303. 6-Amino-2-(3′-bromo-4-{3-[1-(4-trifluoromethyl-benzenesulfonyl)-piperidin-4-yl]-propyl}-3,4,5,6-tetrahydro-2H-[1,2′]bipyridinyl-5′-sulfonylamino)-hexanoic acid

304. 6-Amino-2-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-hexanoic acid

305. 3-(3-Chloro-4-{4-[3-(1-pyridin-2-ylmethyl-piperidin-4-yl)-propyl]-piperidin-1-yl}-benzenesulfonylamino)-propionic acid

306. 6-Amino-2-[4-(4-{3-[I-(4-bromo-5-chloro-thiophene-2-sulfonyl)-piperidin-4-yl]-propyl}-piperidin-1-yl)-3-chloro-benzenesulfonylamino]-hexanoic acid

307. {4-[4-(4-{3-[1-(5-Bromo-6-methoxy-pyridine-3-sulfonyl)-piperidin-4-yl]-propyl}-piperidin-1-yl)-3-chloro-benzenesulfonyl]-[1,4]diazepan-1-yl}-acetic acid

308. {1-[4-(4-{3-[1-(5-Bromo-6-chloro-pyridine-3-sulfonyl)-piperidin-4-yl]-propyl}-piperidin-1-yl)-3-chloro-benzenesulfonyl]-pyrrolidin-3-ylamino}-acetic acid

309. 2-Amino-3-[4-(4-{3-[1-(4-bromo-5-chloro-thiophene-2-sulfonyl)-piperidin-4-yl]-propyl}-piperidin-1-yl)-3-chloro-benzenesulfonylamino]-propionic acid

310. 3-Benzo[1,3]dioxol-5-yl-3-[4-(4-{3-[1-(4-bromo-5-chloro-thiophene-2-sulfonyl)-piperidin-4-yl]-propyl}-piperidin-1-yl)-3-chloro-benzenesulfonylamino]-propionic acid

311. 1-{1-[4-(4-{3-[1-(5-Bromo-6-chloro-pyridine-3-sulfonyl)-piperidin-4-yl]-propyl}-piperidin-1-yl)-3-chloro-benzenesulfonyl]-pyrrolidin-3-yl}-3-ethyl-urea 312. N-{1-[4-(4-{3-[1-(5-Bromo-6-chloro-pyridine-3-sulfonyl)-piperidin-4-yl]-propyl}-piperidin-1-yl)-3-chloro-benzenesulfonyl]-pyrrolidin-3-yl}-methanesulfonamide

313. {1-[4-(4-{3-[1-(5-Bromo-6-chloro-pyridine-3-sulfonyl)-piperidin-4-yl]-propyl}-piperidin-1-yl)-3-chloro-benzenesulfonyl]-pyrrolidin-3-yl}-dimethyl-amine

314. 1-[4-(4-{3-[1-(5-Bromo-6-chloro-pyridine-3-sulfonyl)-piperidin-4-yl]-propyl}-piperidin-1-yl)-3-chloro-benzenesulfonyl]-piperidin-4-ol

315. 1-[4-(4-{3-[1-(4-Bromo-5-chloro-thiophene-2-sulfonyl)-piperidin-4-yl]-propyl}-piperidin-1-yl)-3-chloro-benzenesulfonyl]-piperidine-4-carboxylic acid

316. 3-[4-(4-{3-[1-(5-Bromo-6-chloro-pyridine-3-sulfonyl)-piperidin-4-yl]-propyl}-piperidin-1-yl)-3-chloro-benzenesulfonylamino]-cyclohexanecarboxylic acid

317. 1-[3-Chloro-4-(4-{3-[1-(4-trifluoromethyl-benzenesulfonyl)-piperidin-4-yl]-propyl}-piperidin-1-yl)-benzenesulfonyl]-piperidine-4-carboxylic acid

318. 4-(4-{3-[1-(5-Bromo-6-chloro-pyridine-3-sulfonyl)-piperidin-4-yl]-propyl}-piperidin-1-yl)-3-chloro-benzoic acid

319. (3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-phenyl-acetic acid

B. General Synthetic Scheme

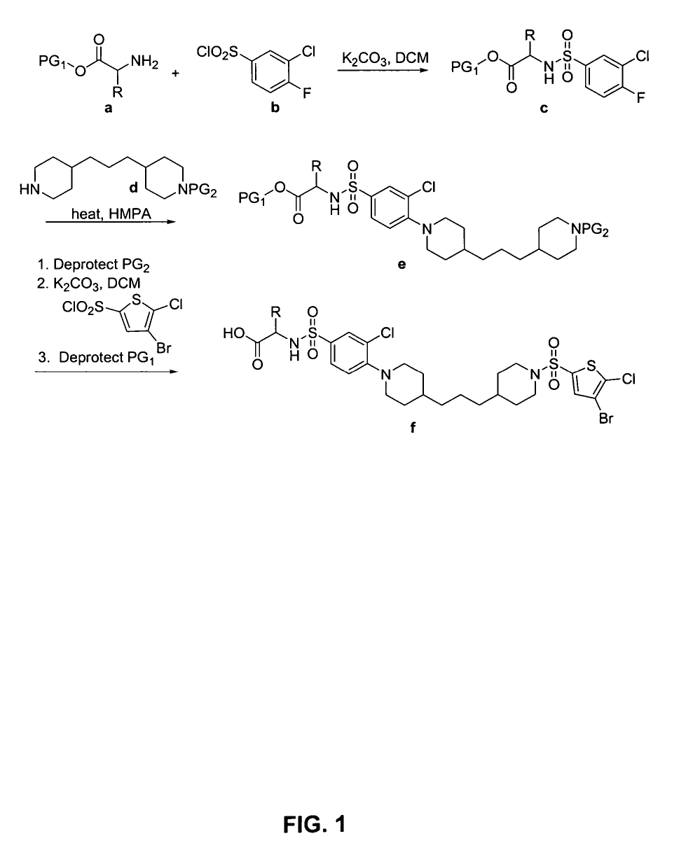

The compounds of the present invention are readily prepared from commercially available starting materials generally following the synthetic scheme set forth in FIG. 1.

With reference to FIG. 1, an acid protected amino acid a is added to a 4-flouro-aryl-sufonyl chloride b under basic conditions at low temperature, for example, at 0° C. (Basrur, V. et al., J. Biol. Chem., 275, 14890-14897). The resultant intermediate c is heated to reflux temperature with a mono protected bis-piperazinyl spacer unit d to provide intermediate e (Jones, J. J., Amino Acid and Peptide Synthesis (Oxford Chemistry Primers, No 7), Oxford University Press, London, 1994). The PG₂ group of e (Terrier, F., Nucleophilic Aromatic Displacement: The Influence of the Nitro Group. John Wiley & Sons; N.Y., 1991) is selectively removed and then reacted with an aryl sulfonyl chloride under basic conditions. Final removal of protecting group 1 (PG₁) yields the final product f. Purification of intermediate e and the final product f is performed by semi prep HPLC (high performance liquid chromatography) produces the compounds of the invention.

One skilled in the art will appreciate that substitution of one starting material or reagent for another in the general synthetic scheme described in FIG. 1 will provide additional compounds within the scope of the present invention. Other starting materials and synthetic routes that can be used in the present invention are described in Examples 1 to 22. Generally, a skilled artisan will recognize that other primary and secondary amines can be used in place of acid protected amino acid a in FIG. 1. In addition, a skilled artisan will recognize other variations in the reaction scheme that will produce the different compounds of the invention. For example, the nucleophilic aromatic substitution reaction (SNAR reaction) used in step 2 of FIG. 1 is facilitated by when the aryl group, for example 3, has electron withdrawing substituent(s). Alternatively, when the aryl group contains electron donating substituents, a skilled artisan will recognize other synthetic methodologies (i.e., Palladium(0) mediated coupling reaction), that can be applied to formed the desired product. Other variations on the synthetic scheme described in FIG. 1 will be apparent to one skilled in the art.

C. Pharmaceutical Compositions

The present invention further provides compositions comprising one or more of the above compounds in combination with a pharmaceutically acceptable excipient. The present invention also provides compositions comprising one or more of the above compounds in combination with at least one additional HIV active agent and a pharmaceutically acceptable excipient.

In one embodiment, the present invention provides a compound of formula I combined with a pharmaceutically acceptable excipient such as sterile saline or other medium, water, gelatin, an oil, etc. to form pharmaceutically acceptable compositions. The compositions and/or compounds may be administered alone or in combination with any convenient carrier, diluent, etc. and such administration may be provided in single or multiple dosages. Useful carriers include, but are not limited to, solid, semi-solid or liquid media including water and non-toxic organic solvents.

In another embodiment, the present invention provides the compounds of formula I in the form of a pro-drug, which can be metabolically or chemically converted to the subject compound by the recipient host. A wide variety of pro-drug derivatives are known in the art such as those that rely on hydrolytic cleavage or oxidative activation of the prodrug.

The compositions can be provided in any convenient form, including tablets, capsules, lozenges, troches, hard candies, powders, sprays, creams, suppositories, etc. As such, the compositions, in pharmaceutically acceptable dosage units or in bulk, may be incorporated into a wide variety of containers. For example, dosage units may be included in a variety of containers including capsules, pills, etc.

The pharmaceutical compositions of the present invention are suitable for use in a variety of drug delivery systems. Examples of suitable formulations for use in the present invention are found in Remington's Pharmaceutical Sciences (Mack Publishing Company, Philadelphia, Pa., 17th ed. (1985)), which is incorporated herein by reference. In addition, for a brief review of methods for drug delivery, see, Langer, Science 249:1527-1533 (1990), which is incorporated herein by reference.

The pharmaceutical compositions of the present invention are intended for parenteral, topical, oral or local administration. In certain aspects, the pharmaceutical compositions are administered parenterally, e.g., intravenously, subcutaneously, intradermally, or intramuscularly. In one embodiment, the invention provides compositions for parenteral administration which comprise a compound of the present invention, dissolved or suspended in an acceptable carrier, preferably an aqueous carrier. A variety of aqueous carriers may be used including, for example, water, buffered water, 0.4% saline, 0.3% glycine, hyaluronic acid and the like. These compositions may be sterilized by conventional, well-known sterilization techniques or, they may be sterile filtered. The resulting aqueous solutions may be packaged for use as is or lyophilized, the lyophilized preparation being combined with a sterile solution prior to administration. The compositions may contain pharmaceutically acceptable auxiliary substances as required to approximate physiological conditions including pH adjusting and buffering agents, tonicity adjusting agents, wetting agents and the like, such as, for example, sodium acetate, sodium lactate, sodium chloride, potassium chloride, calcium chloride, sorbitan monolaurate, triethanolamine oleate, etc.

For solid formulations, compounds of the present invention can be admixed with conventional nontoxic solid carriers can be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient and more preferably at a concentration of 25%-75%.

For aerosol administration, the compounds of the present invention and antidiabetic agents are preferably supplied in finely divided form along with a surfactant and propellant. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. A carrier can also be included, as desired, as with, e.g., lecithin for intranasal delivery.

The compounds of the present invention can be prepared and administered in a wide variety of oral and parenteral dosage forms. Thus, the compounds of the present invention can be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. Accordingly, the present invention also provides pharmaceutical compositions comprising a pharmaceutically acceptable carrier or excipient and either a compound of the present invention or a pharmaceutically acceptable salt.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances that may also act as diluents, flavoring agents, binders, preservatives, tablet disintegrating agents, or an encapsulating material.

In powders, the carrier is a finely divided solid that is in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1000 mg, preferably 1.0 mg to 100 mg according to the particular application and the potency of the active component. The composition can, if desired, also contain other compatible therapeutic agents.

It will be appreciated that the actual preferred course of therapy will vary according to, inter alia, the mode of administration of the compound of the present invention, the particular formulation being utilized, the mode of administration of the compounds, the particular disease being treated and the particular host being treated. The optimal course of therapy for a given set of conditions can be ascertained by those skilled in the art using conventional course of therapy determination tests and in view of the information set out herein.

III. Method of Use

The present invention provides methods of treating infections (i.e., vial, bacterial), by administering to a subject having such a disease or condition, a therapeutically effective amount of a compound of the present invention. In a preferred embodiment the infection is a HIV infection and AIDS. The “subject” is defined herein to include animals such as mammals, including, but not limited to, primates (e.g., humans), cows, sheep, goats, horses, dogs, cats, rabbits, rats, mice and the like.

Depending on the subject's condition, the compounds of the present invention may be administered by oral, parenteral (e.g., intramuscular, intraperitoneal, intravenous, ICV, intracistemal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration.

In certain aspects, in the treatment or prevention of conditions which require modulation of the CD4/GP120 interaction, an appropriate dosage level will generally be about 0.001 to 100 mg per kg patient body weight per day which can be administered in single or multiple doses. In one embodiment the dosage level will be about 0.01 to about 50 mg/kg per day. In another embodiment the dosage level will be preferably about 0.05 to about 25 mg/kg per day. A suitable dosage level may be about 0.01 to 50 mg/kg per day or about 0.01 to 25 mg/kg per day or about 0.01 to 10 mg/kg per day. For oral administration, the compositions are preferably provided in the form of tablets containing 1.0 to 1500 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, 1000.0, 1100.0, 1200.0, 1300.0, 1400.0 and 1500.0 milligrams of the active ingredient for the symptomatic adjustment of the dosage to the patient to be treated. The compounds can be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The present invention is also directed to combinations of the compounds with one or more agents having related utilities to prevent and treat HIV infectivity and AIDS. For example, the compounds of the inventions may be effectively administered, whether at periods of pre-exposure or post-exposure, in combination with effective amounts of HIV antiviral agents, anti-infective agents and immunomodulators having as described but not limited to those agents listed in Tables 1-3. Additionally, the compounds of the invention may be used in combination with other HIV entry inhibitors which are discussed further in Drugs of the Future 1999, 24(12), pp. 1355-1362; Cell, Vol. 9, pp. 243-246, Oct. 29, 1999; and Drug Discovery Today, Vol. 5, No. 5, May 2000, pp. 183-194.

The present compounds are administered as part of a monotherapy, a combination therapy, triple therapy, or quadruple therapy program. Preferably, the present compounds are used as part of a combination therapy program.

The compounds of the present invention is used in combination with one or more antiviral agents selected from the group consisting of: 097, Amprenivir 141 W94 GW 141, Abacavir (1592U89) GW 1592, Acemannan, Acyclovir, AD-439, AD-519, Adefovir dipivoxil, AL-72 1, Alpha Interferon, Ansamycin LM 427, Antibody which Neutralizes pH Labile alpha aberrant Interferon, AR177, Beta-fluoro-ddA, BMS-232623 (CGP-73547), BMS-234475 (CGP-61755), CI-1012, Cidofovir, Curdlan sulfate, Cytomegalovirus Immune globin, Cytovene Ganciclovir, Delaviridine, Dextran Sulfate, ddC Dideoxycyfidine, ddl Dideoxyinosine, DMP-450, Efavirenz (DMP 266)(-)6-Chloro-4-(S)-cyclopropylethynyl-4(S)-trifluoro-methyl-1,4-dihydro-2H-3,1-benzoxazin-2-one, STOCRINE, EL10, Famciclovir, FTC, GS 840, HBY097, Hypericin, Recombinant Human Interferon Beta, Interferon alfa-n3, Indinavir, ISIS 2922, KNI-272, Lamivudine, 3TC, Lobucavir, Nelfinavir, Nevirapine, Novapren, Peptide T Octapeptide Sequence, Trisodium Phosphonoformate, PNU-440690, Probucol, RBC-CD4, Ritonavir, Saquinavir, Stavudine; d4T Didehydrodeoxy-thymidine, Valaciclovir, Virazole Ribavirin, VX-478, Zalcitabine, Zidovudine; AZT.

In another embodiment, the compounds of the present invention are used in combination with one or more immunomodulators selected from the group consisting of: AS-101, Bropirimine, Acemannan, CL246,738, EL10, FP-21399, Gamma Interferon, Granulocyte Macrophage Colony Stimulating Factor, Granulocyte Macrophage Colony Stimulating Factor, Granulocyte Macrophage Colony Stimulating Factor, HIV Core Particle Immunostimulant, IL-2 Interleukin-2, JL-2 Interleukin-2, IL-2 Interleukin-2 (aldeslukin), Immune Globulin Intravenous (human), IMREG-1, IMREG-2, Imuthiol Diethyl Dithio Carbamate, Alpha-2 Interferon, Methionine-Enkephalin, MTP-PE Muramyl-Tripeptide, Granulocyte Colony Stimulating Factor, Remune, rCD4 Recombinant Soluble Human CD4, rCD4-IgG hybrids, Recombinant Soluble Human CD4, Interferon Alfa 2a, SK & F 106528 Soluble T4, Thymopentin, Tumor Necrosis Factor; TNF.

In yet another embodiment, the compounds of the present invention are used in combination with one or more anti-infectives selected from the group consisting of: Clindamycin with Primaquine, Fluconazole, Pastille Nystatin Pastille, Omidyl Eflornithine, Pentamidine Isethionate (IM & IV), Trimethoprim, Trimethoprim/sulfa, Piritrexim, Pentamidine Isethionate for Inhalation, Spiramycin, Intraconazole-R51211, Trimetrexate, Daunorubicin, Recombinant Human Erythropoietin, Recombinant Human Growth Hormone, Megestrol Acetate, Testosterone, Total Enteral Nutrition.

Preferably, the compounds of the present invention is used in combination with one or more anti-HIV agents for example: nucleoside analogue reverse transcriptase inhibitors (NRTIs) (i.e., abacavir (Ziagen®), didanosine (Videx®), lamivudine (Epivir®), stavudine (Zerit®), zalcitabine (Hivid®), zidovudine (Retrovir®), zidovudine/lamivudine/abacavir (Trizivir®), protease inhibitors (PIs) (i.e., amprenavir (Agenerase®), indinavir (Crixivan®), lopinavir/ritonavir (Kaletra®), ritonavir (Norvir®), nelfinavir (Viracept®), saquinavir (Fortovase®), atazanavir (Reyataz®), nonnucleoside reverse transcriptase inhibitors (nNRTIs) (i.e., delavirdine (Rescriptor®), efavirenz (Sustiva®), nevirapine (Viramune®), capravirine, amdoxivir, nucleotide reverse transcriptase inhibitors such as tenofovir (Viread®), integrase inhibitors (GlaxoSmithKline/Shionogi's S-1360).

In another preferred embodiment, the present compounds may also be used in conjunction with one or more HIV entry inhibitors for example: fusion inhibitors (i.e., Trimeris/Roche's T-20/pentafuside (Fuzeon®, T-1249), co-receptor antagonists such as CCR5 and CXCR4 antagonists (i.e., Progenic's PRO-1 40, Anormed's AMD-3100, Schering-Plough's SCH-C and SCH-D, Takeda's TAK779, Allelix's ALX40-4C) and attachment inhibitors (i.e., Progenic's PRO-542, BMS-488043).

In another embodiment, the present compounds may be administered in conjuction was a second non-HIV therapeutic active agent, for example, an anticancer drug such as the ribonucleotide reductase inhibitor, hydroxyurea (Hydrea).

In the above combinations, the compounds of the present invention and other HIV active agents may be administered separately or in conjunction. In addition, the administration of one agent may be prior to, concurrent with, or subsequent to the administration of the other agents.

IV. Examples

A. In Vitro Assays

1. Preparation of Recombinant gp120 and CD4

Method 1:

Coding sequences for gp120 (macrophage-tropic strain JR-FL) and human CD4 (comprised of amino acids 1-371) were sub-cloned into the commercial baculovirus expression vector pAcGP67 (Pharmingen, San Diego, Calif.). The recombinant vectors were then transfected into Sf9 insect cell monolayers using the Baculogold™ transfection system. Clones were isolated by the method of limited dilution.

For large-scale production, cell culture was scaled up in 8-liter spinner flasks of Sf9 cells adapted to serum-free growth conditions, then infected by baculovirus using an MOI of 5 and the cell supernatant was harvested 72 hours later. The yields of recombinant protein expressed for both proteins were similar; 2-5 μg/ml (micrograms per milliliter). Recombinant CD4 protein was immunopurified using an affinity-column of immobilized Anti-CD4 D1 mAb Leu3a (Dalgleish, A. G., et al., Lancet, 2:1047-1050 1987) and gp120 protein was immunopurified using a resin coupled with Propharmacon monoclonal antibody PPC3G6 produced in-house (mouse IgG2) against Sf9-produced gp120. Protein purity and molecular weight were assessed using reduced and non-reduced SDS PAGE.

Method 2:

Using M-tropic strain JR-FL and CD4 (1-370) cloned into baculovirus expression vectors, PCR (polymerase chain reaction) is used to introduce the coding sequences from the above plasmid into a new vector which will introduce a (His)₆ tail on the COOH terminus of the protein. Alternatively, (His)₆ tails is added on the current baculovirus constructs. One new suitable vector, pIZ/V5-His can be purchased from Invitrogen, and would allow creation of a stable line in Sf9 cells. Cloning is accomplished by PCR of the coding sequences with flanking oligos oligonucleotides encoding chosen restriction sites (suitable for insertion within the multiple cloning site of the plasmid) for each end of the gene that would be ligated into the appropriately cut vector. Directional cloning of the insert using two different restriction enzymes is chosen. Alternatively, if the final construct yielded too little protein or is unsuccessful at producing a stable line, the (His)₆ tails or another purification tag is then incorporated on the current baculovirus constructs. This is accomplished first by locating two restriction sites, one up-stream of the stop codon and one down-stream, and excising this fragment with the two restriction enzymes. A fragment generated by PCR from the intact template and two oligos duplicating the two restriction sites but with the downstream oligo adding sequence for a (His)₆ tail and a new stop codon is restriction digested, annealed, and ligated. In this case, re-establishment of a protein expression system entails transfection of plasmids into Sf9 cells, amplification of viral stocks, and scale-up growth and infection in cells grown under serum-free conditions to ease purification.

Similar to the above, the recombinant T-tropic strain Bru/LAV gp120 is prepared following one of the above procedures for the protein expression of LAV. More specifically, a PCR-amplified fragment from a publicly available clone that is flanked with preferably two different restriction sites is restriction digested, annealed and ligated into an appropriately cut vector.

The purification step of cell-clarified, Sf9 supernatants is done using commercial nickel-nitrilotriacetic acid (NTA) resin. Standard protocols will be followed for the chromatography of proteins, with purity and quantitation being assessed by SDS PAGE and a BCA protein determination kit (Pierce). Further chromatographic purification using size exclusion or anion/cation exchange may optionally be carried out as needed if high purity is not attained with one NTA purification step.

2. Binding Assay

Method 1:

The identification of inhibitors was carried out by a competition assay utilizing recombinant macrophage-tropic strain JR-FL gp120, which competes with a prospective inhibitor to bind to immobilized recombinant CD4. The amount of gp12O binding to CD4 was quantitated by DELFIA “Dissociation Enhanced Lanthanide Fluoroimmunoassay” (Paterson, N., et al., Ann. Clin Biochem., 22, 606-611, 1985), having previously labeled the gp120 with a commercial europium chelate (Wallac Oy, Turku, Finland). The final readout uses time-resolved fluorescence to quantitate the amount of dissociated europium metal present. Binding of labeled gp120 was saturable and yielded an IC₅₀ of 3 nM, which approximates literature values (Lasky, L. A., et al., Cell, 50, 975-985). In addition, the binding of gp120 was inhibited by Leu3a with an IC₅₀ of 2 μg/ml. Appropriate control compounds such as aurintricarboxylic acid (ATA) and cosalane were utilized in optimizing the binding assay and they produced IC₅₀'s close to cited values, 0.5 and 4.3 μg/ml, respectively (Schols, D. et al., Proc. Natl. Acad. Sci. USA, 86, 3322-3326, 1989); Cushman, M. et al., J. Med. Chem., 37, 3040-50, 1994). The assay is optionally adapted to 384-well format for screening purposes.

Optionally, the compounds of the invention may be assessed for inhibitory activity against T-lymphotropic strains (e.g. LAV, IIIB) of HIV by using a modified format of the above described competition assay. In the modified assay, the T-tropic gp120 LAV (Protein Sciences Corp., Meriden, Conn.), when bound to immobilized CD4, was detected by a mouse anti-gp120 antibody (Intracel, Cockeysville, Md.) followed by a europium-labeled rabbit anti-mouse antibody (Wallac Oy).

Method 2:

The competition assay to study the effects of test compounds on the binding of gp120 to immobilized CD4 can also be assessed using an ELISA assay (Enzyme-Linked Immunosorbent Assay). In this assay, the soluble CD4 purified from Sf9 supernatants is immobilized to 96-well microtiter plates by an overnight incubation in bicarbonate buffer at 4° C. The plate is then washed, blocked, and gp120 added and incubated for a suitable time at room temperature. The plate is then washed and a volume of anti-gp120 monoclonal antibody-horseradish peroxidase conjugate added. After incubation, the plates are washed with a horseradish peroxidase-conjugated second antibody. Optionally, an anti-gp120 monoclonal such as NEA 9205 (NEN DuPont) is substituted for the conjugate, followed by addition of a horseradish peroxidase-conjugated anti-mouse IgG1 antibody. Finally, the plates are washed and developed with ortho phenylenediamine (OPD) substrate and read spectrophotometrically. The dose-response curve of the gp120 will yield a half-maximal binding value (IC50). Cosalane and ATA will be used as standard positive controls. Optionally, the compound are also screen in the presence of mucin.

3. Cell Fusion Assay

A cell-cell fusion assay described in the literature (Nussbaum, O. et al., J. Virol., 68, 5411-22, 1994) was adopted to evaluate the gp120/CD4 antagonists for their ability to inhibit Env-mediated cell fusion. This assay generally requires transfection of target cells (PA317) with a reporter gene plasmid that is activated upon fusion with gp120-expressing effector cells (HeLa). The system that was used employed a vaccinia virus vector carrying the Env gene (dual tropic gp120 89.6/gp41) as well as T7 polymerase to infect the effector cells. The target cells were transfected with genes for CD4 and CCR5, and the T7 promoter linked to a luciferase reporter gene. Upon gp120/CD4-based cell fusion, effector cell T7 polymerase is capable of binding the target cell's T7 promoter, which drives luciferase production. Cell fusion was allowed to proceed for 5.5 hours with compound present throughout the incubation and 10% FBS (fetal bovine serum) was present. The final read-out was made with Bright-Glo™ reagent (Promega, Madison, Wis.) after cell lysis.

4. Cytotoxicity

There are several reagents available to quantitate cytotoxicity of organic compounds. These include tetrazolium reagents such as MTT, XTT, MTS (Promega), and Alamar blue. All these reagents rely on metabolic reduction in living cells resulting in the production of a colored or fluorescent product. Any of these reagents would suffice for quantitation of cell proliferation and toxicity and there are only slight advantages of one over another. The Alamar blue dye has the advantage it can be read both fluorescently or spectrophotometrically. The MTS reagent has a benefit in that it contains an additive that converts the insoluble formazan product to a soluble one that eliminates the acidic solubilization steps needed for MTT. Both Alamar blue and MTS allow repeated sample reading over time for additional color development rather than a single endpoint reading.

### EXAMPLES

Chemistry: Unless otherwise noted, all solvents and reagents were obtained from commercial suppliers and used without further purification. Analytical thin layer chromatography (TLC) was performed on aluminum sheets precoated with silica gel obtained from Merck. Visualization was accomplished by using an UV light (254 nm) after dipping in phosphomolybdic acid in MeOH followed by heating. Purification was done by semi-preparative HPLC on a Ranin HPLX instrument with a YMC 2.5 μm C18 analytic column (50×4.6 mm, (10-95% ACN/H₂O 0.1% TFA, 7 min, 3 mL/min)) and a two-channel UV detector (220 and 260 nm). Infrared spectra (IR) were recorded in the range of 4000 cm⁻¹ to 600 cm⁻¹ using a Perkin-Elmer Spectrum BX Fourier transformed infrared spectrophotometer. Infrared spectra of solids were obtained from Nujol mull samples and liquids samples were applied neat on sodium chloride disks. UV spectra were obtained using a HP 8425 UV/VIS instrument with quartz 1 mL cuvets. ¹H and ¹³C NMR experiments were performed on a Bruker AMX 400 MHz instrument. Chemical shifts are reported in parts per million (ppm) relative to tetramethylsilane (TMS). Low-resolution ESI-MS mass spectra were recorded on a Finnigan LCQ DUO MS instrument using a Gilson analytic HPLC with UV/VIS detector. APCI (atmospheric chemical ionization) mass spectrometry was performed on a Finnigan TSQ 7000 Triple Quad MS instrument equipped with a HP 1090 HPLC. Purity of compounds was checked with a reversed phase Ranin SD200 analytic HPLC with PDA detector or a Waters 2690 analytic HPLC with model 996 PDA detector and both equipped with a YMC 3.5 μm C 18 analytic column (20×4.6 mm, UV detection (10-90% ACN/H₂O 0.1% TFA, 12 min, 220 nm, 1 mL/min)). The following chemical reagents are abbreviated as follows:
DMF=dimethylforamide
DIEA=diisopropyethylamine
DCM=dichloromethane
Et2O=diethylether
FMOC=fluorenylmethyoxycarbonyl
TFA=trifluoroacetic acid
MeOH=Methanol
EtOAc=Ethyl acetate

### Example 1

The following example illustrates the synthesis of 2-(3′-Bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonylamino)-4-methylsulfanyl-butyric acid (29).

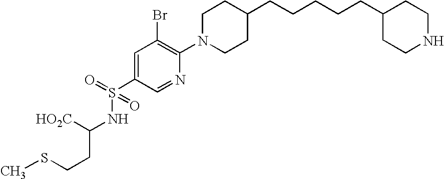

2-[3′-Bromo-4-(5-piperidin-4-yl-pentyl)-3,4,5,6-tetrahydro-2H-[1,2′]bipyridinyl-5′-sulfonylamino]-4-methylsulfanyl-butyric acid: To a 25 mL sterile centrifuge tube was added 5 mL of DCM. To this was added 1.0 g of Irori Wang resin (1.36 mmol/g loading), 1.2 mL DMF, 1.5 mL of DIEA. The resin was allowed to pre-swell for 20 Minutes. 1.5 g of N-FMOC-2-Amino-2-(S-methyl-2-ethyl sulfide)-ethanoic acid (Met Amino Acid) was added and the solution was capped and gently agitated for 20 hours. A small sample of the resin (10.8 mg) was washed (2×DMF, 2×DCM, 2×Et₂O) and dried in a vacuum for 30 minutes. To this resin was added 1 mL of 30% piperdine in DMF for 30 minutes to remove the FMOC protecting group. 1 mL of DMF was added and four 100 L samples were analyzed by UV to determine the FMOC content. The loading was determined to be 1.0 mmol/g (92% of theoretical). In addition, a small sample was washed as above and the compound cleaved off with 1 mL of 90% TFA in DCM for 30 minutes. A single peak on the HPLC was observed at 6.01 min, which corresponds to the methionine amino acid (Met AA). The resin was added to 20 mL of 30% piperdine/DMF solution for one hour, washed (2×DMF, 2×DCM, 2×Et₂O) and dried in a vacuum for 12 hours. To a 10 mL sterile centrifuge tube was added 2.5 mL of DCM, 276 μL of DIEA, and 0.5 g of the Met AA resin; the resin was left to stand for 20 minutes. To this was added the 182 mg of 5-Bromo-6-chloro-pyridine-3-sulfonyl chloride dissolved in 2.5 mL of DCM and 111 uL of DIEA. After 2 hours the resin was rinsed with 2×5 mL DCM, 2×5mL DMF, 2×5 mL MeOH, 1×5mL EtOAc and 2×5 mL Et₂O followed by the addition of bromophenyl blue solution to 0.3 mg of beads. The primary amine starting material was not detected. In addition, a small sample was washed as above and the compound cleaved off with 1 mL of 90% TFA in DCM for 30 minutes. MS (ES⁻) m/z 402 (M−H); HPLC (214 nm) rt 5.31 min 99.0%. To a 10 mL sterile centrifuge tube was added 3 mL of NMP, 221 μL of DIEA, and 0.25 g of the pyridine sulfonamide Met AA resin; the resin was left to stand for 20 minutes. To this was added the 143 mg of 1,5-(4-piperdine)pentane and the solution heated at 70° C. for 15 hours. After cooling, the resin was rinsed with 2×5 mL DCM, 2×5 mL DMF, 2×5 mL MeOH, 2×5 mL DCM, 1×5 mL EtOAc and 2×5 mL Et₂O. In addition, a small sample was washed as described above and the compound cleaved off with 1 mL of 90% TFA in DCM for 30 minutes. MS (ES⁻) m/z 604 (M−H); HPLC (214 nm), retention time (rt) 5.62 min, 97.0% purity.

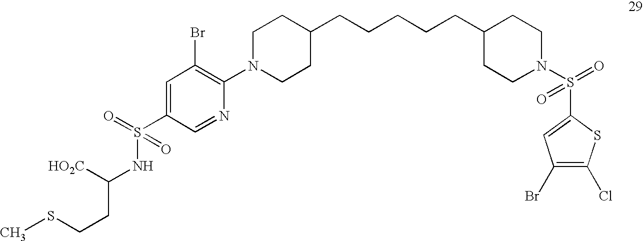

2-(3′-Bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonylamino)-4-methylsulfanyl-butyric acid (29): To a 10 mL sterile centrifuge tube was added 3 mL of DCM and 0.25 g of 2-[3′-Bromo-4-(5-piperidin-4-yl-pentyl)-3,4,5,6-tetrahydro-2H-[1,2′]bipyridinyl-5′-sulfonylamino]-4-methylsulfanyl-butyric acid. After 20 minutes of standing 111 μL of DIEA and 89 mg of 4-Bromo-5-chloro-thiophene-2-sulfonyl chloride were added. After 2 hours, the resin was washed with 2×5 mL DCM, 2×5 mL DMF, 2×5 mL MeOH, 2×5 mL DCM, 1×5 mL EtOAc and 2×5 mL Et₂O. Cleavage of the product from the resin was accomplished by the addition of 5 mL 90% TFA in DCM to the resin-bound product in a 15 mL enclosed fritted funnel and stirring for 30 minutes. The filtrate was removed, the resin washed with 3×15 mL DCM, and all fractions collected. Rotary evaporation of the filtrate left 15 mg of a crude product, an oil (88% crude yield). The crude oil was purified by semi-prep HPLC to obtain 6.2 mg (36.5% yield) of a white fluffy powder after lyophilization. ¹H NMR 400 MHz CDCl₃ δ_{H} 8.57 (1H s), 8.05 (1H s), 7.23 (1H s), 5.41 (1H, br s), 4.16 (1H br s), 2.06 (3H s), 4.14-1.18 (33H m); MS (ES⁻) m/z 865 (M−H); HPLC (214 nm), rt 8.81 min, 98.9% purity.

### Example 2

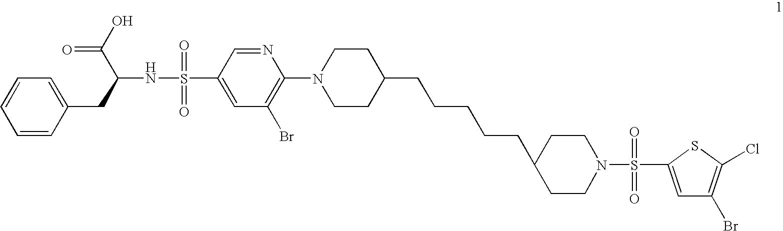

2-(3′-Bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonylamino)-3-phenyl-propionic acid (1): ¹H NMR 400 MHz CDCl₃ δ_{H} 8.41 (1H, s), 7.87(1H, s), 7.31 (1H, s), 7.32-7.04 (SH, m), 5.08-1.09 (33, m); MS (ES⁺) m/z 881 (M+H); HPLC (214 nm), rt 8.35 min, 98.7% purity.

### Example 3

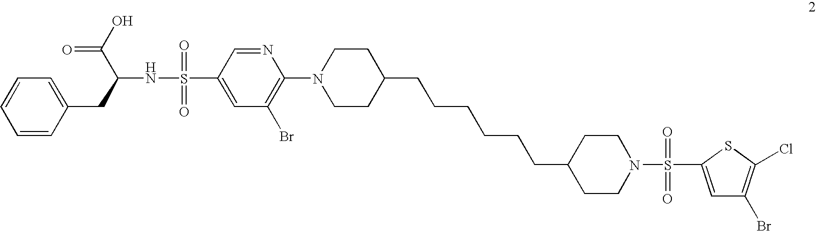

2-(3′-Bromo-4-{6-[1-(4-bromo-5-chloro-thiophene-2-sulfonyl)-piperidin-4-yl]-hexyl}-3,4,5,6-tetrahydro-2H-[1,2′]bipyridinyl-5′-sulfonylamino)-3-phenyl-propionic acid (2): ¹H NMR 400 MHz CDCl₃ δ_{H} 8.40 (1H, s), 7.77(1H, s), 7.28 (1H, s), 7.28-7.05 (5H, m), 5.19-1.17 (35, m); MS (ES⁺) m/z 895 (M+H); HPLC (214 nm), rt 8.61 min, 95.2% purity.

### Example 4

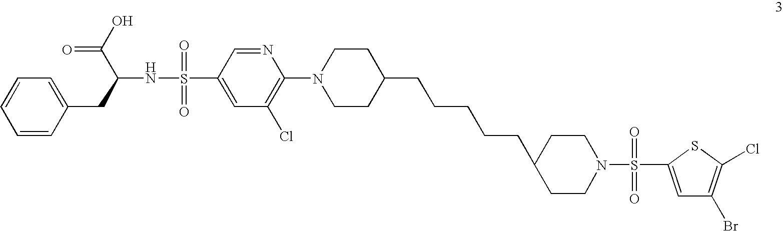

2-(4-{5-[1-(4-Bromo-5-chloro-thiophene-2-sulfonyl)-piperidin-4-yl]-pentyl}-3′-chloro-3,4,5,6-tetrahydro-2H-[1,2′]bipyridinyl-5′-sulfonylamino)-3-phenyl-propionic acid (3): ¹H NMR 400 MHz CDCl₃ δ_{H} 8.38 (1H, s), 7.76(1H, s), 7.30 (1H, s), 7.29-7.06 (5H, m), 5.10-1.15 (33, m); MS (ES⁺) m/z 837 (M+H); HPLC (214 nm), rt 8.31 min, 99.7% purity.

### Example 5

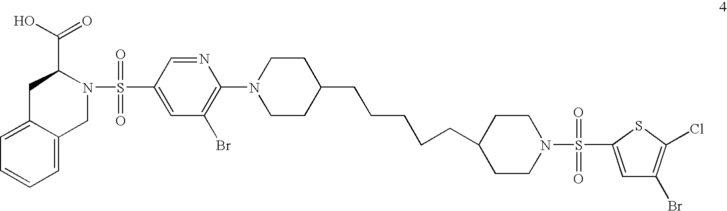

2-(3′-Bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonyl)-1,2,3,4-tetrahydro-isoquinoline-3-carboxylic acid (4): ¹H NMR 400 MHz CDCl₃ δ_{H} 8.50 (1H, s), 8.08(1 H, s), 7.30 (1H, s), 7.25-7.01 (5H, m), 5.02-1.18 (33, m); MS (ES⁺) m/z 893 (M+H); HPLC (214 nm), rt 8.55 min, 96.9% purity.

### Example 6

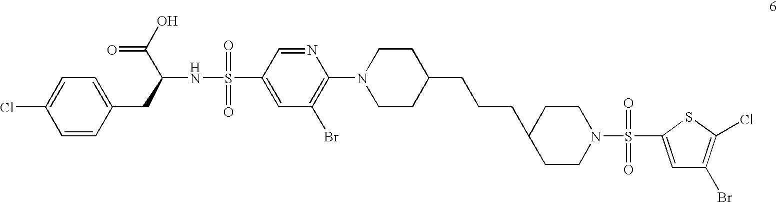

2-(3′-Bromo-4-{3-[1-(4-bromo-5-chloro-thiophene-2-sulfonyl)-piperidin-4-yl]-propyl}-3,4,5,6tetrahydro-2H-[1,2′]bipyridinyl-5′-sulfonylamino)-3-(4-chloro-phenyl)-propionic acid (6): ¹H NMR 400 MHz CDCl₃ δ_{H} 8.36 (1H, s), 7.89 (1H, s), 7.36 (1H, s), 7.28-7.05 (4H, m), 5.20-1.19 (29, m); MS (ES⁺) m/z 887 (M+H); HPLC (214 nm), rt 8.04 min, 98.7% purity.

### Example 7

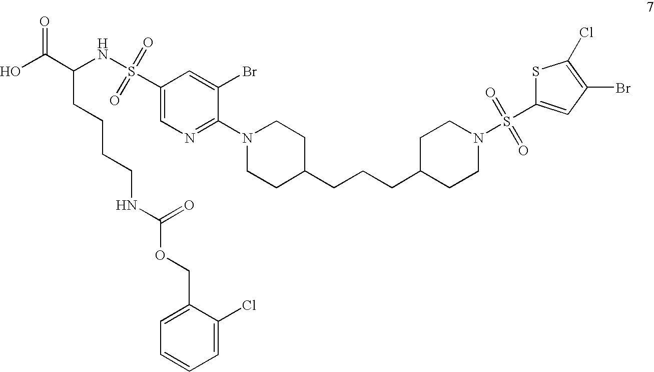

2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-6-(2-chlorobenzyloxycarbonylamino)-hexanoic acid (7): ¹H NMR 400 MHz CD₃OD δ_{H} 8.48 (1H, s), 8.13 (1H, s), 7.69-7.25 (6H m), 5.18 (2H, s), 4.06-1.21 (36H m); MS (ES⁺) m/z 1000 (M+H); HPLC (214 nm), rt 9.52 min, 99.1% purity.

### Example 8

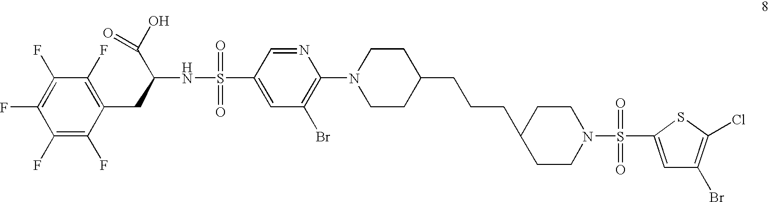

2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-pentafluorophenyl-propionic acid (8): ¹H NMR 400 MHz CDCl_{3 δ}_{H} 8.37 (1H, s), 7.88 (1H, s), 7.32 (1H, s), 5.19-1.20 (29, m); MS (ES⁺) m/z 943 (M+H); HPLC (214 nm), rt 8.01 min, 99.2% purity.

### Example 9

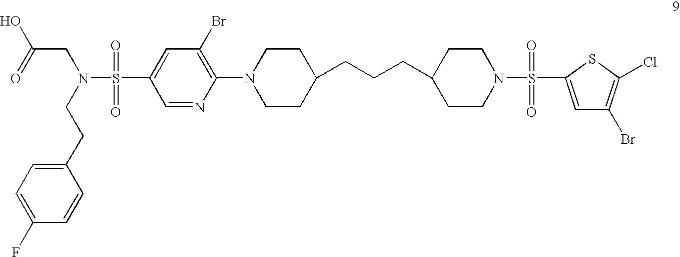

{(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-[2-(4-fluoro-phenyl)-ethyl]-amino}-acetic acid (9): ¹H NMR 400 MHz CD₃OD δ_{H} 8.49 (1H s), 7.98 (1H s), 7.28 (1H s), 7.11 (2H d, J=10.4 Hz), 6.95 (2H d, J=10.6 Hz), 4.12-1.21 (31H m); MS (ES⁺) m/z 885 (M+H); HPLC (214 nm), rt 9.53 min, 99.8% purity.

### Example 10

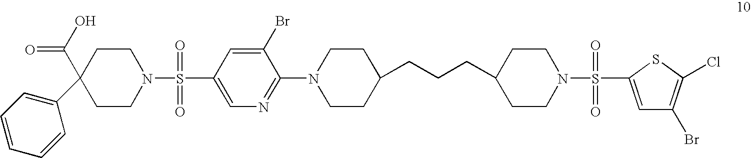

1-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-4-phenyl-piperidine-4-carboxylic acid (10): ¹H NMR 400 MHz CDCl_{3 δ}_{H} 8.51 (1H, s), 8.00 (1H, s), 7.31 (1H, s), 7.30-7.19 (5H, m), 4.14-1.18 (33, m); MS (ES⁺) m/z 893 (M+H); HPLC (214 nm), rt 8.31 min, 95.6% purity.

### Example 11

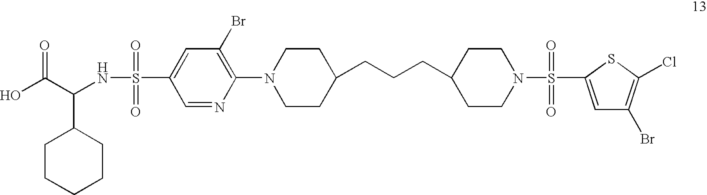

(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-cyclohexyl-acetic acid (13): ¹H NMR 400 MHz CD₃OD δ_{H} 8.44 (1H s), 8.12 (1H s), 7.53 (1H s), 2.90 (3H s), 4.01-1.04 (37H m); MS (ES⁺) m/z 845 (M+H); HPLC (214 nm), rt 9.53 min, 97.8% purity.

### Example 12

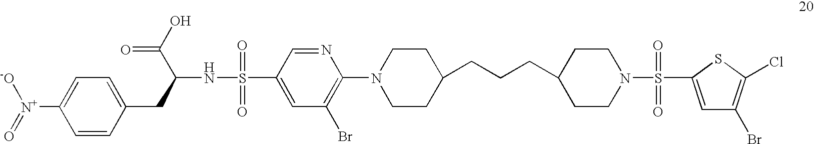

2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(4-nitro-phenyl)-propionic acid (20): ¹H NMR 400 MHz CDCl₃ δ_{H} 8.39 (1H, s), 7.92 (1H, s), 7.30 (1H, s), 7.72-7.33 (4H, m), 4.23-1.22 (29, m); MS (ES⁺) m/z 893 (M+H); HPLC (214 nm), rt 8.31 min, 95.6% purity.

### Example 13

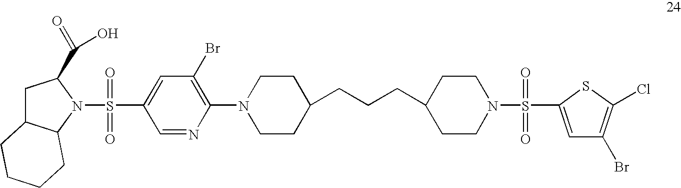

1-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonyl)-octahydro-indole-2-carboxylic acid (24): ¹H NMR 400 MHz CD₃OD δ_{H} 8.40 (1H s), 8.11 (1H s), 7.37 (1H s), 4.08-1.05 (37H m); MS (ES⁺) m/z 858 (M+H); HPLC (214 nm), rt 8.24 min, 99.3% purity.

### Example 14

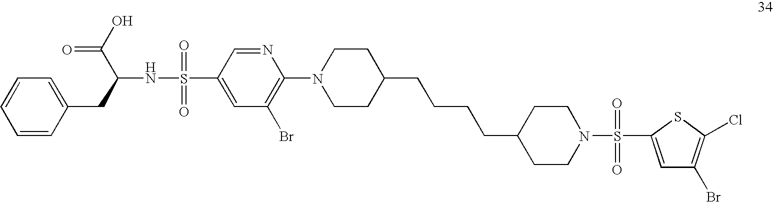

2-(3′-Bromo-4-{4-[1-(4-bromo-5-chloro-thiophene-2-sulfonyl)-piperidin-4-yl]-butyl}-3,4,5,6-tetrahydro-2H-[1,2′]bipyridinyl-5′-sulfonylamino)-3-phenyl-propionic acid (34): ¹H NMR 400 MHz CDCl₃ δ_{H} 8.42 (1H, s), 7.87(1H, s), 7.30 (1H, s), 7.31-7.07 (5H, m), 5.08-1.07 (31, m); MS (ES⁺) m/z 867 (M+H); HPLC (214 nm), rt 8.11 min, 95.1% purity.

### Example 15

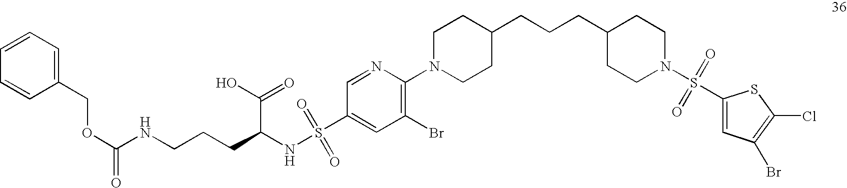

5-Benzyloxycarbonylamino-2-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-pentanoic acid (36): ¹H NMR 400 MHz dmso-d₆ δ_{H} 8.42 (1H, s), 8.24 (1H, s), 8.05(1H, s), 7.72 (1H s), 7.33 (5H br s), 4.91 (2H, s), 3.97-1.04 (33H m); MS (ES⁺) m/z 954 (M+H); HPLC (214 nm), rt 8.26 min, 99.6% purity.

### Example 16

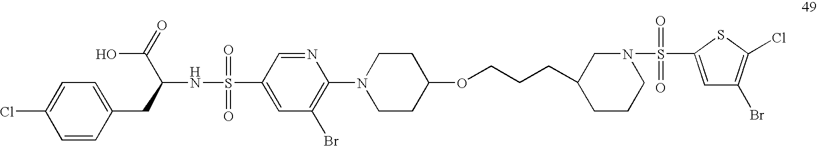

2-(3′-Bromo-4-{3-[1-(4-bromo-5-chloro-thiophene-2-sulfonyl)-piperidin-3-yl]-propoxy}-3,4,5,6-tetrahydro-2H-[1,2′]bipyridinyl-5′-sulfonylamino)-3-(4-chlorophenyl)-propionic acid (49): ¹H NMR 400 MHz CDCl₃ δ_{H} 8.38 (1H, s), 7.93 (1H, s), 7.31 (1H, s), 7.28-7.06 (4H, m), 5.19-1.20 (29, m); MS (ES⁺) m/z 904 (M+H); HPLC (214 nm), rt 7.71 min, 99.7% purity.

### Example 17

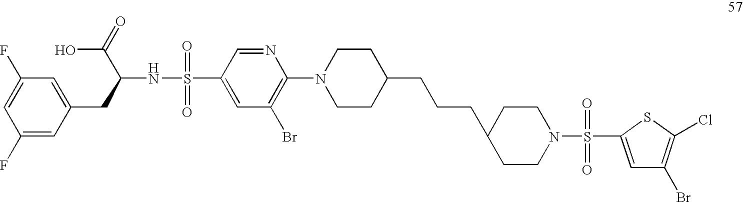

2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(3,5-difluorophenyl)-propionic acid (57): ¹H NMR 400 MHz CDCl₃ δ_{H} 8.46 (1H, s), 7.90 (1H, s), 7.30 (1H, s), 6.72-6.60 (3H, m), 5.33-1.19 (29, m); MS (ES⁺) m/z 889 (M+H); HPLC (214 nm), rt 7.86 min, 99.6% purity.

### Example 18

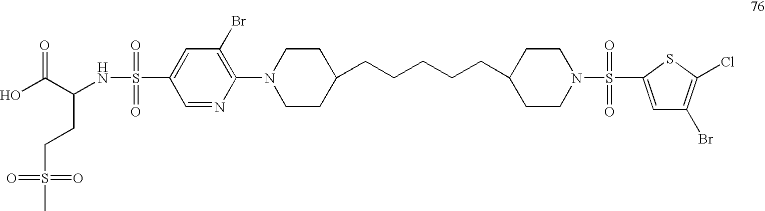

2-(3′-Bromo-4-{5-[1-(4-bromo-5-chloro-thiophene-2-sulfonyl)-piperidin-4-yl]-pentyl}-3,4,5,6-tetrahydro-2H-[1,2′]bipyridinyl-5′-sulfonylamino)-4-methanesulfonylbutyric acid (76): ¹H NMR 400 MHz CDCl₃ δ_{H} 8.47 (1H s), 8.03 (1H s), 7.27 (1H s), 2.90 (3H s), 4.09-1.06 (35H m); MS (ES⁺) m/z 897 (M+H); HPLC (214 nm), rt 9.52 min, 99.7% purity.

### Example 19

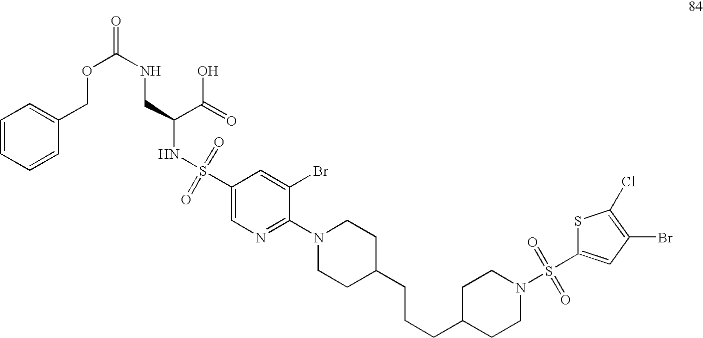

3-Benzyloxycarbonylamino-2-(3′-bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-propionic acid (84): ¹H NMR 400 MHz CD₃OD δ_{H} 8.49 (1 H, s), 8.17 (1 H, s), 7.52 (1H s), 7.27 (5H br s), 5.09-1.10 (36H m); MS (ES⁺) m/z 926 (M+H); HPLC (214 nm), rt 9.52 min, 99.5% purity.

### Example 20

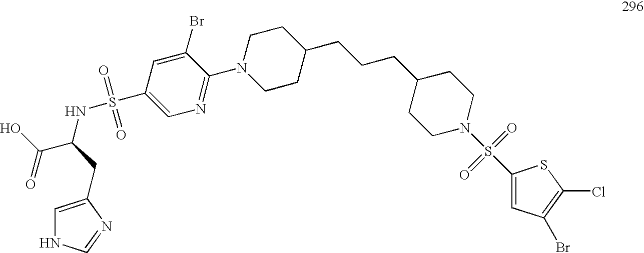

2-(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-3-(1H-imidazol-4-yl)-propionic acid (296): ¹H NMR 400 MHz CD₃OD δ_{H} 8.82 (1H s),8.44 (1H, s), 8.41 (1H, s), 8.12 (1H s), 8.09 (1H s), 8.06 (1H s), 7.50 (1H s), 7.42 (1H s), 4.29-1.07 (23H m); MS (ES⁺) m/z 843 (M+H); HPLC (214 nm), rt 9.52 min, 94.7% purity.

### Example 21

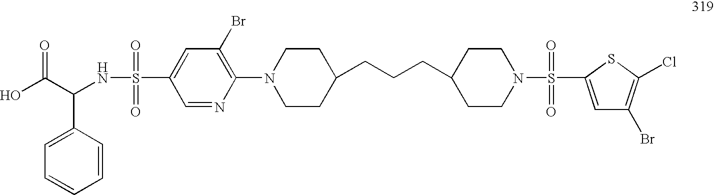

(3′-Bromo-4-{3-[1-(4-bromo-5-chloro-thiophene-2-sulfonyl)-piperidin-4-yl]-propyl}-3,4,5,6-tetrahydro-2H-[1,2′]bipyridinyl-5′-sulfonylamino)-phenyl-acetic acid (319): ¹H NMR 400 MHz CD₃OD δ_{H} 8.70 (1H s),8.49 (1H, s), 8.35 (1H, s), 7.88 (1H s), 7.21 (5H br s), 5.01 (1H s), 3.99-1.16 (24H m); MS (ES⁺) m/z 837 (M+H); HPLC (214 nm), rt 9.53 min, 99.7% purity.

### Example 22

The compounds in Table 2 were prepared using the procedure outlined in Example 1. These compounds were tested in the gp120/CD4 binding assay described above and exhibited the following levels of activity: +, IC50>10 μM; ++, IC50<10 μM and >1 μM; +++IC50<1 μM.

TABLE II

| Compound | Activity |
|---|---|
| 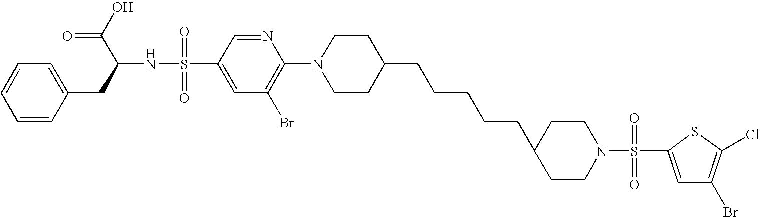 | +++ |
| 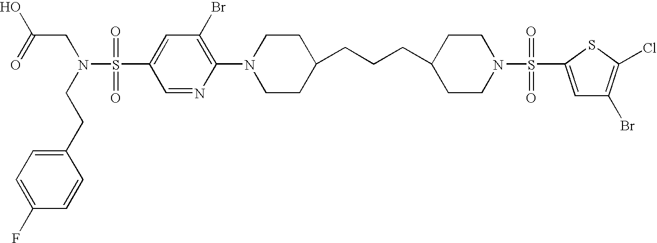 | +++ |
| 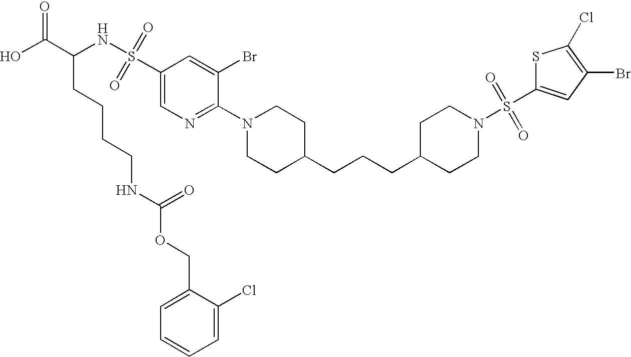 | +++ |
| 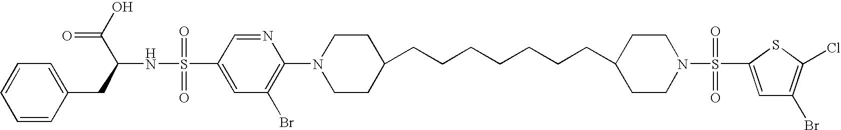 | +++ |
| 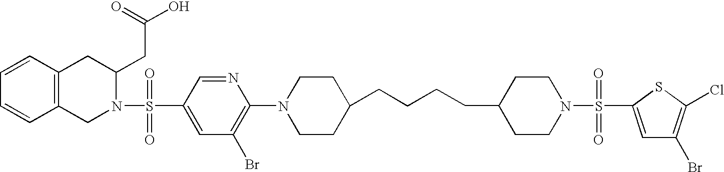 | +++ |
| 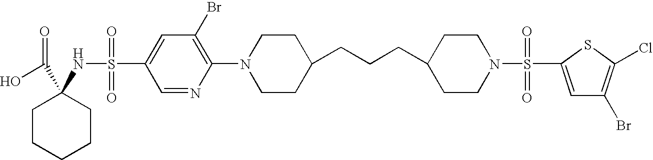 | +++ |
| 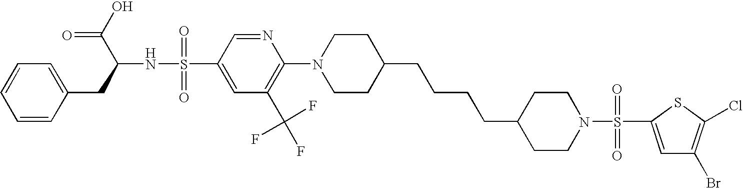 | ++ |
| 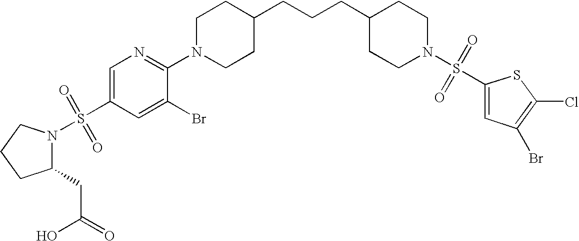 | ++ |
| 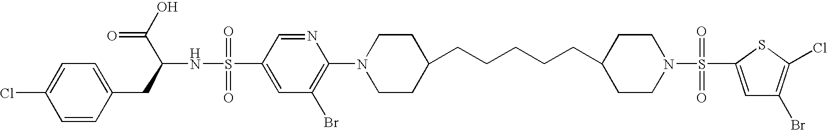 | ++ |
| 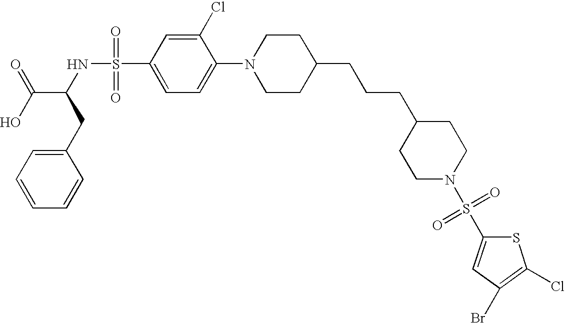 | ++ |
| 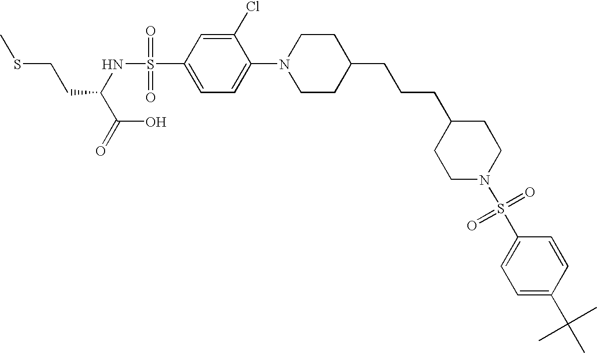 | ++ |
| 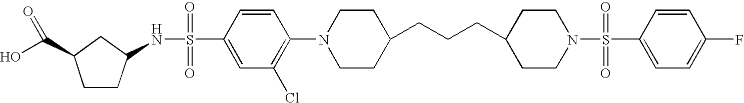 | + |
| 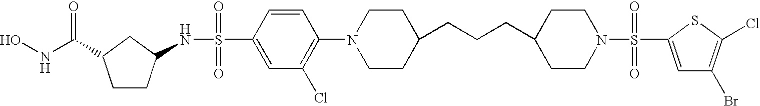 | + |
| 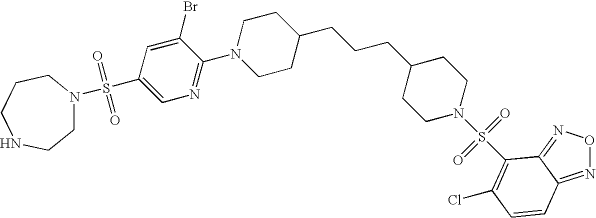 | + |

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A compound of formula I: 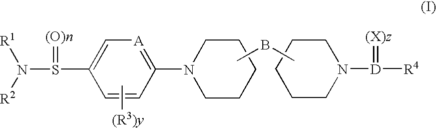
and pharmaceutically acceptable salts thereof;
wherein
R¹ and R² are each independently a member selected from the group consisting of hydrogen, alkyl, cycloalkyl, heterocycloalkyl, arylalkyl, heteroalkyl, heterocycloalkyl, hetrocycloalkylalkyl, heteroarylalkyl and —CH₂CO₂H;
alternatively R¹ and R² together with the nitrogen atom to which they are attached join to form a 4-to 7-membered heterocyclic ring optionally containing 1-3 additional heteroatoms as ring members and optionally substituted with members selected from the group consisting of alkyl, heteroalkyl, aryl, heteroaryl, hydroxy, halogen, —CO₂R¹¹ wherein R¹¹ is hydrogen or (C₁-C₄)alkyl, and alkoxy, wherein if present, any of said substituents located on adjacent atoms in said 4-to 7-membered ring may optionally be replaced with a substituent of formula -E-(CH₂)ᵤ—F— to form a fused ring wherein,
u is an integer from 1-2;
E and F are each independently CH₂, O or NH;
wherein up to three bonds in said fused ring formed may optionally be replaced with a double bond; and
wherein said fused ring formed may further be substituted with 0-4 substitutents selected from the group consisting of halogen, haloalkyl, alkyl, aryl, —CN and —NO₂;
n and z may be the same or different and are integers from 1-2;
R³ is a member selected from the group consisting of halogen, alkyl, heteroalkyl, halo(C₁-C₄)alkyl, —S(O)₂R¹³, —S(O)R¹³ and —NO₂, wherein R¹³ is selected from the group consisting of (C₁-C₄)alkyl, alkylamino and amino;
y is an integer from 0-4;
A is N or C;
B is (C₁-C₁₀)alkylene or (C₁-C₁₀)heteroalkylene;
D is C or S;
X, if present, is O, N or S; and
R⁴ is selected from the group consisting of aryl and heteroaryl.

2. The compound of claim 1, wherein at least one of R¹ and R² is further substituted with —CO₂R¹⁰, wherein R¹⁰ is (C₁-C₄)alkyl or hydrogen.

3. The compound of claim 1, wherein R⁴ is selected from the group consisting of phenyl, furanyl, benzofuranyl, pyridyl, thienyl and benzothienyl.

4. The compound of claim 1, wherein said compound of formula I has formula II 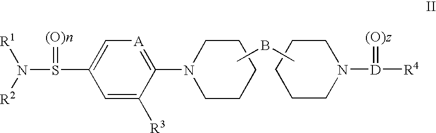
wherein R³ is selected from the group consisting of chloro, bromo and trifluoromethyl.

5. The compound of claim 1, wherein [symbol] -NR¹R² is selected from the group consisting of 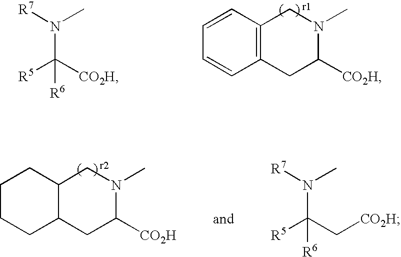
wherein the subscripts r1 and r2 are each an integer from 0-1;
R⁵, R⁶ and R⁷ are independently selected from the group consisting of hydrogen, alkyl, cycloalkyl, heteroalkyl, aryl, heteroaryl, arylalkyl, heteroarylalkyl; or R⁵ and R⁶ together with the carbon atom to which they are attached join to form a 4-to 7-membered ring optionally having heteroatoms as ring members and optionally substituted with members selected from the group consisting of alkyl, heteroalkyl, aryl, heteroaryl, hydroxy, halogen, —C(O)₂H and alkoxy, wherein if present, any of said substituents located on adjacent atoms in said 4-to 7-membered ring may optionally be replaced with a substituent of formula -E-(CH₂)ᵤ—F— to form a fused ring wherein;
u is an integer from 1-2;
E and F are each CH₂;
wherein up to 3 bonds in said fused ring formed may optionally be replaced with a double bond; and
wherein said fused ring formed may further be substituted with 0-4 substituents selected from the group consisting of halogen, halo(C₁-C₄)alkyl, alkyl, —CN and —NO₂.

6. The compound of claim 5, wherein [symbol] -NR¹R² is selected from the group consisting of 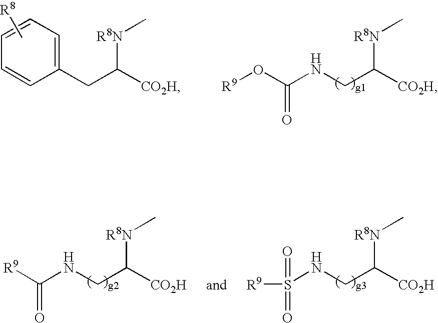
wherein the subscripts g1 to g3 are each an integer from 1-4;
R⁸ is selected from the group consisting of hydrogen, halogen, alkyl, heteroalkyl, aryl, heteroaryl, arylalkyl, heteroarylalkyl, halo(C₁-C₄)alkyl, hydroxyl, alkoxy and —NO₂; and
R⁹ is selected from the group consisting of alkyl, heteroalkyl, aryl, heteroaryl, arylalkyl, heteroarylalkyl and halo(C₁-C₄)alkyl.

7. The compound of claim 1, wherein B is (C₂-C₇)alkylene or (C₂-C₇)heteroalkylene.

8. The compound of claim 1, wherein A is nitrogen.

9. The compound of claim 1, wherein R³ is halogen or halo(C₁-C₄)alkyl; and y is an integer from 1-2.

10. The compound of claim 1, wherein z is 2, D is S, and X is O.

11. The compound of claim 1, wherein R⁴ is selected from the group consisting of:

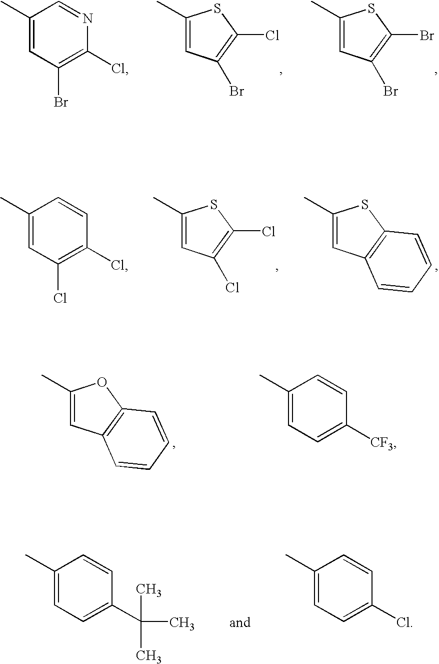

12. The compound of claim 1, wherein R⁴ is selected from the group consisting of: 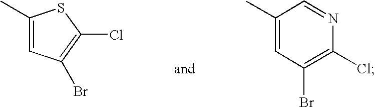
D is S and z is 2.

13. The compound of claim 1, wherein said compound is selected from the group consisting of the compounds in Table I.

14. The compound of claim 13, wherein said compound is selected from the group consisting of:

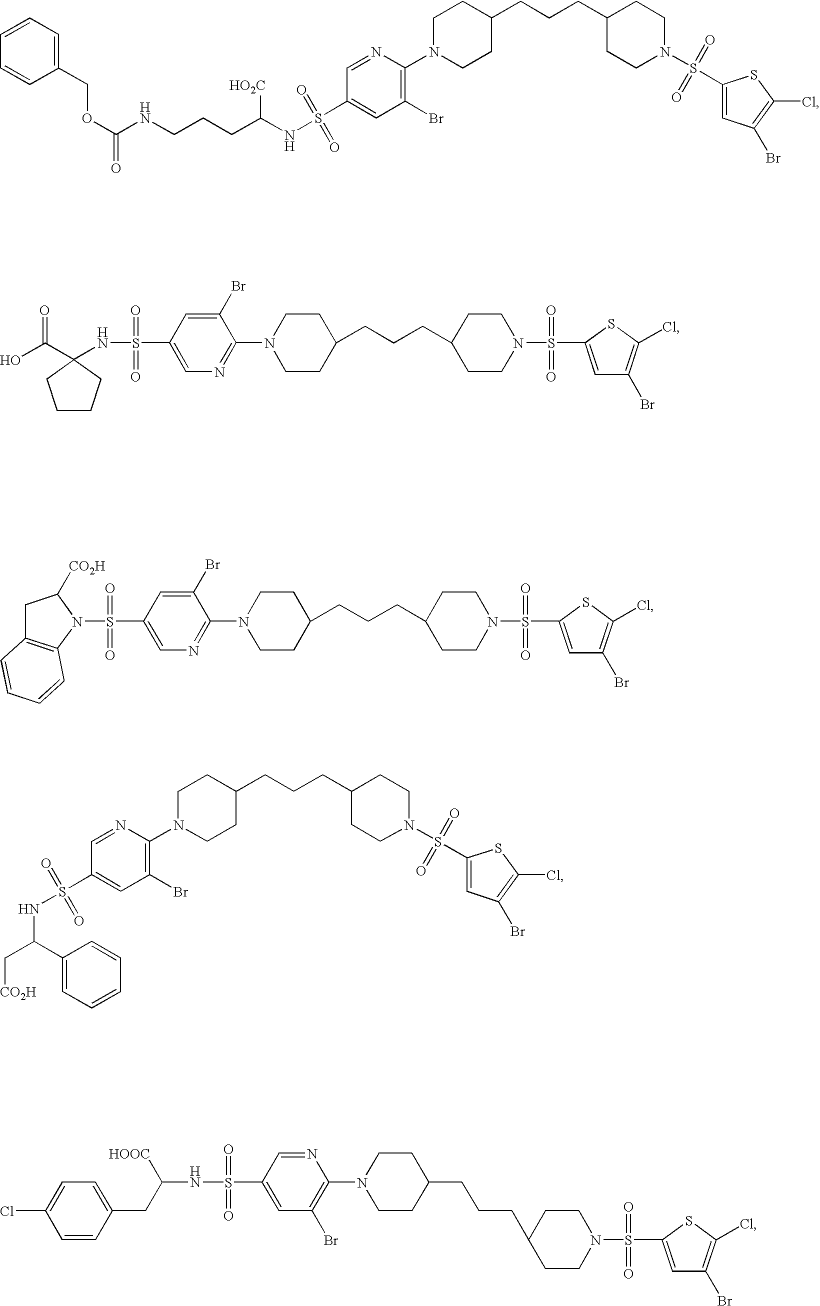

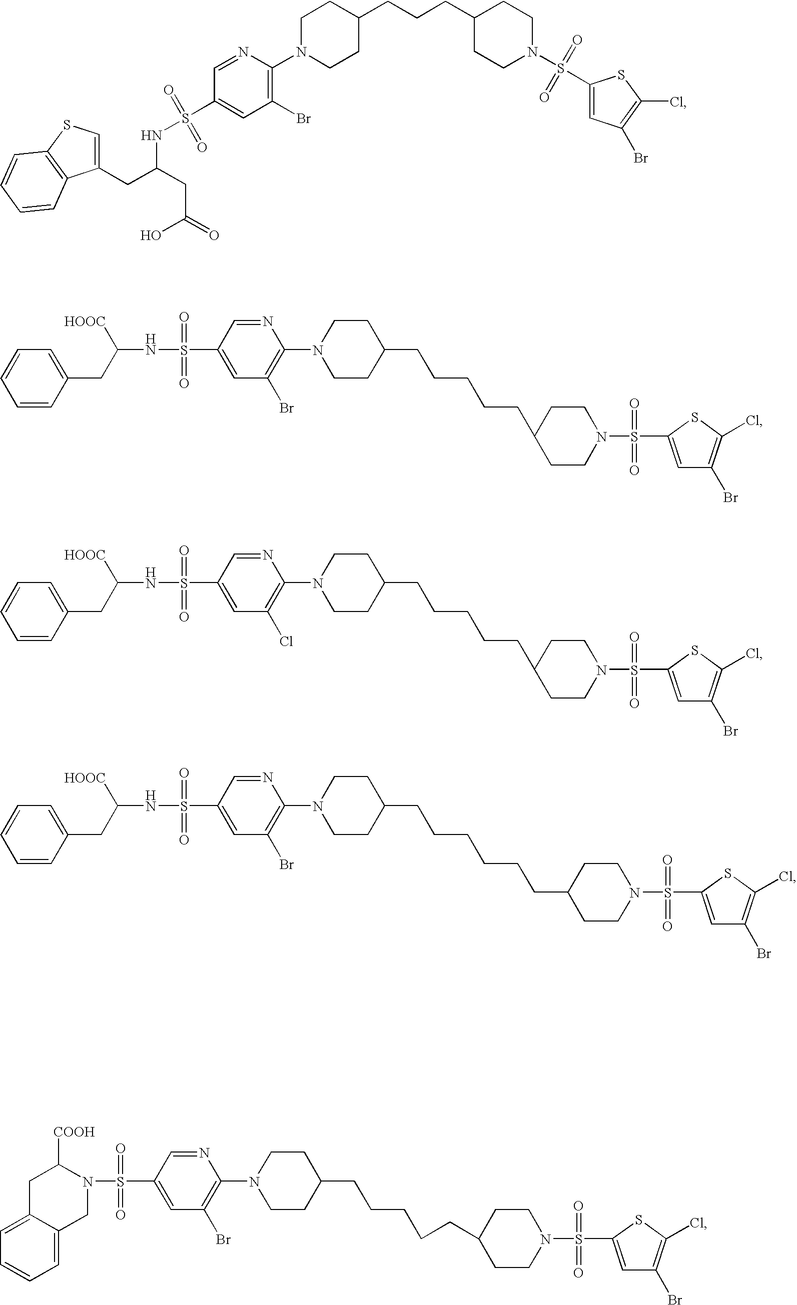

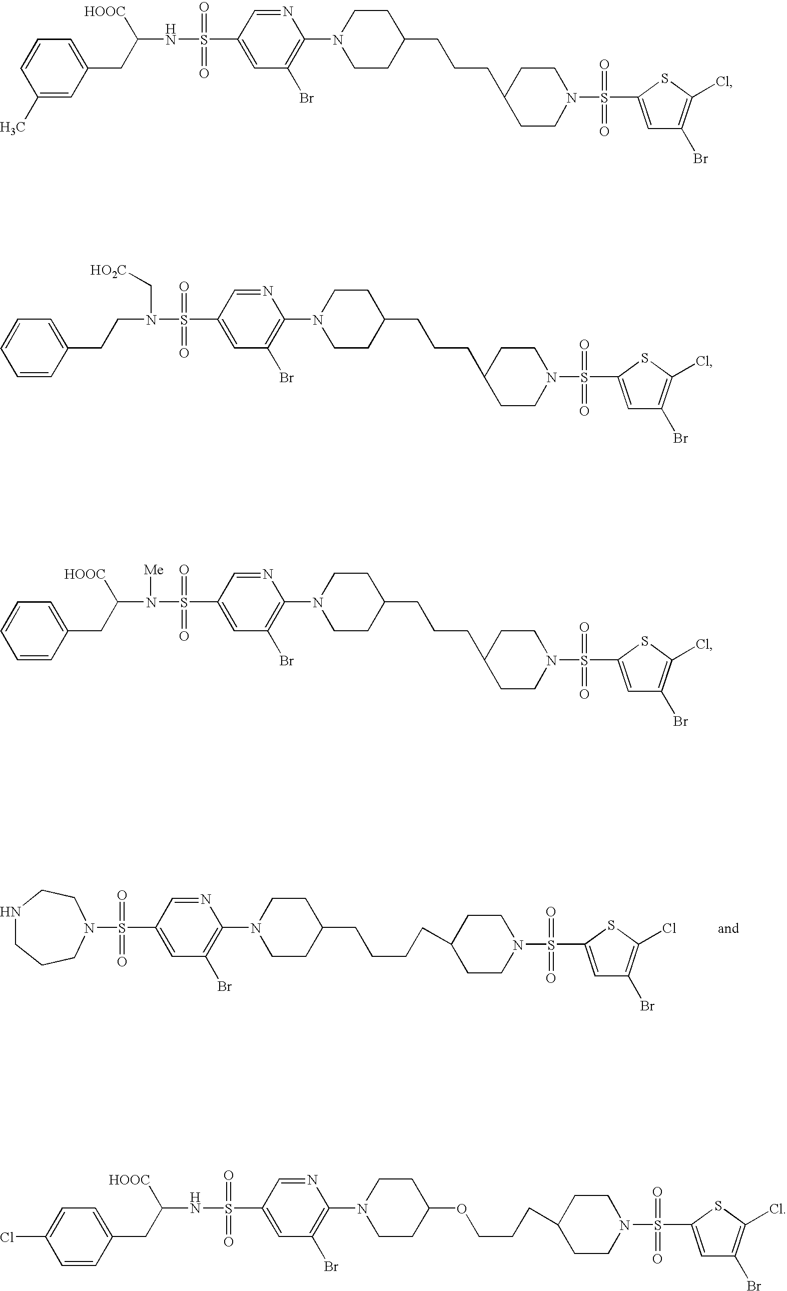

15. A pharmaceutical composition, said composition comprising a pharmaceutically acceptable carrier or excipient and a compound having the formula 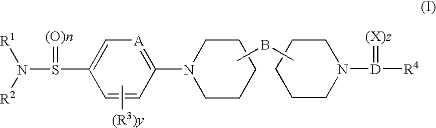
and pharmaceutically acceptable salts thereof;
wherein
R¹ and R² are each independently a member selected from the group consisting of hydrogen, alkyl, cycloalkyl, heterocycloalkyl, arylalkyl, heteroalkyl, heterocycloalkyl, hetrocycloalkylalkyl, heteroarylalkyl and —CH₂CO₂H; alternatively R¹ and R² together with the nitrogen atom to which they are attached, form a 4-to 7-membered heterocyclic ring optionally containing 1-3 additional heteroatoms as ring members and optionally substituted with members selected from the group consisting of alkyl, heteroalkyl, aryl, heteroaryl, hydroxy, halogen, —CO₂R¹¹ wherein R¹¹ is hydrogen or (C₁-C₄)alkyl, and alkoxy, wherein if present, any of said substituents located on adjacent atoms in said 4-to 7-membered ring may optionally be replaced with a substituent of formula -E-(CH₂)ᵤ—F— to form a fused ring wherein,
u is an integer from 1-2;
E and F are each independently CH₂, O or NH;
wherein up to three bonds in said fused ring formed may optionally be replaced with a double bond; and
wherein said fused ring formed may further be substituted with 0-4 substitutents selected from the group consisting of halogen, haloalkyl, alkyl, aryl, —CN and —NO₂;
n and z may be the same or different and are integers from 1-2;
R³ is a member selected from the group consisting of halogen, alkyl, heteroalkyl, halo(C₁-C₄)alkyl, —S(O)₂R¹³, —S(O)R¹³ and —NO₂, wherein R¹³ is selected from the group consisting of (C₁-C₄)alkyl, alkylamino and amino;
y is an integer from 0-4;
A is N or C;
B is (C₁-C₁₀)alkylene or (C₁-C₁₀)heteroalkylene;
D is C or S;
X, if present, is O, N or S; and
R⁴ is selected from the group consisting of aryl and heteroaryl.

16. The composition of claim 15, wherein at least one of R¹ and R² is further substituted with —CO₂R¹⁰, wherein R¹⁰ is (C₁-C₄)alkyl or hydrogen.

17. The composition of claim 15 wherein R⁴ is selected from the group consisting of phenyl, furanyl, benzofuranyl, pyridyl, thienyl and benzothienyl.

18. The composition of claim 15, wherein said compound of formula I has formula II 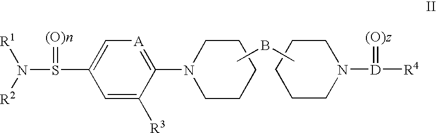
wherein R³ is selected from the group consisting of chloro, bromo and trifluoromethyl.

19. The composition of claim 15, wherein [symbol] -NR¹R² is selected from the group consisting of 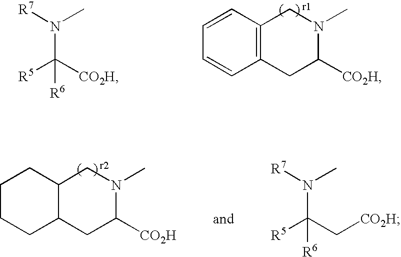
wherein the subscripts r1 and r2 are each an integer from 0-1;
R⁵, R⁶ and R⁷ are independently selected from the group consisting of hydrogen, alkyl, cycloalkyl, heteroalkyl, aryl, heteroaryl, arylalkyl, heteroarylalkyl; or R⁵ and R⁶ together with the carbon atom to which they are attached join to form a 4-to 7-membered ring optionally having heteroatoms as ring members and optionally substituted with members selected from the group consisting of alkyl, heteroalkyl, aryl, heteroaryl, hydroxy, halogen, —C(O)₂H and alkoxy, wherein if present, any of said substituents located on adjacent atoms in said 4-to 7-membered ring may optionally be replaced with a substituent of formula -E-(CH₂)ᵤ—F— to form a fused ring wherein;
u is an integer from 1-2;
E and F are each CH₂;
wherein up to 3 bonds in said fused ring formed may optionally be replaced with a double bond; and
wherein said fused ring formed may further be substituted with 0-4 substituents selected from the group consisting of halogen, halo(C₁-C₄)alkyl, alkyl, —CN and —NO₂.

20. The composition of claim 19, wherein [symbol] -NR¹R² is selected from the group consisting of 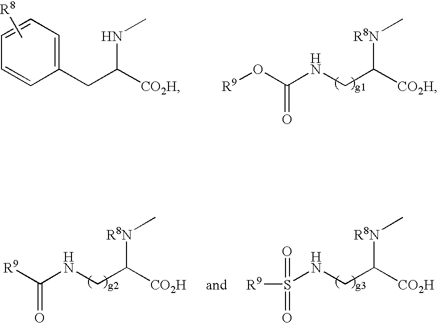
wherein the subscripts g1 to g3 are each an integer from 1-4;
R⁸ is selected from the group consisting of halogen, alkyl, heteroalkyl, aryl, heteroaryl, arylalkyl, heteroarylalkyl, halo(C₁-C₄)alkyl, hydroxyl, alkoxy and —NO₂; and
R⁹ is selected from the group consisting of alkyl, heteroalkyl, aryl, heteroaryl, arylalkyl, heteroarylalkyl and halo(C₁-C₄)alkyl.

21. The composition of claim 15, wherein B is (C₂-C₇)alkylene or (C₂-C₇)heteroalkylene.

22. The composition of claim 15, wherein A is nitrogen.

23. The composition of claim 15, wherein R³ is halogen or halo(C₁-C₄)alkyl; and y is an integer from 1-2.

24. The composition of claim 15, wherein z is 2, D is S, and X is O.

25. The composition of claim 15, wherein R⁴ is selected from the group consisting of:

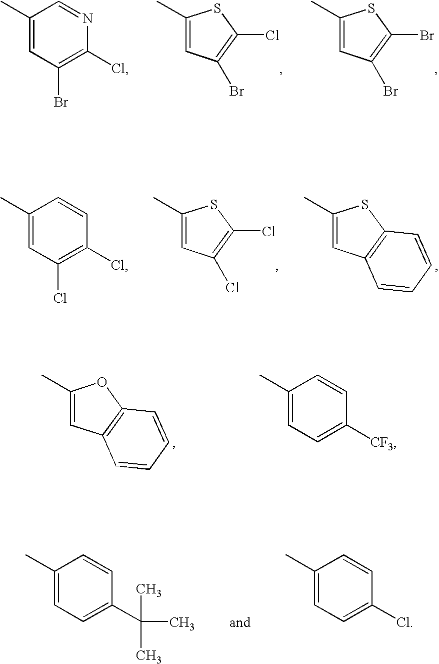

26. The composition of claim 15, wherein R⁴ is selected from the group consisting of: 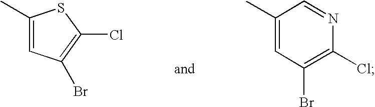
D is S, and z is 2.

27. The composition of claim 15, wherein said compound is selected from the group consisting of the compounds in Table I.

28. The composition of claim 15, wherein said compound is selected from the group consisting of:

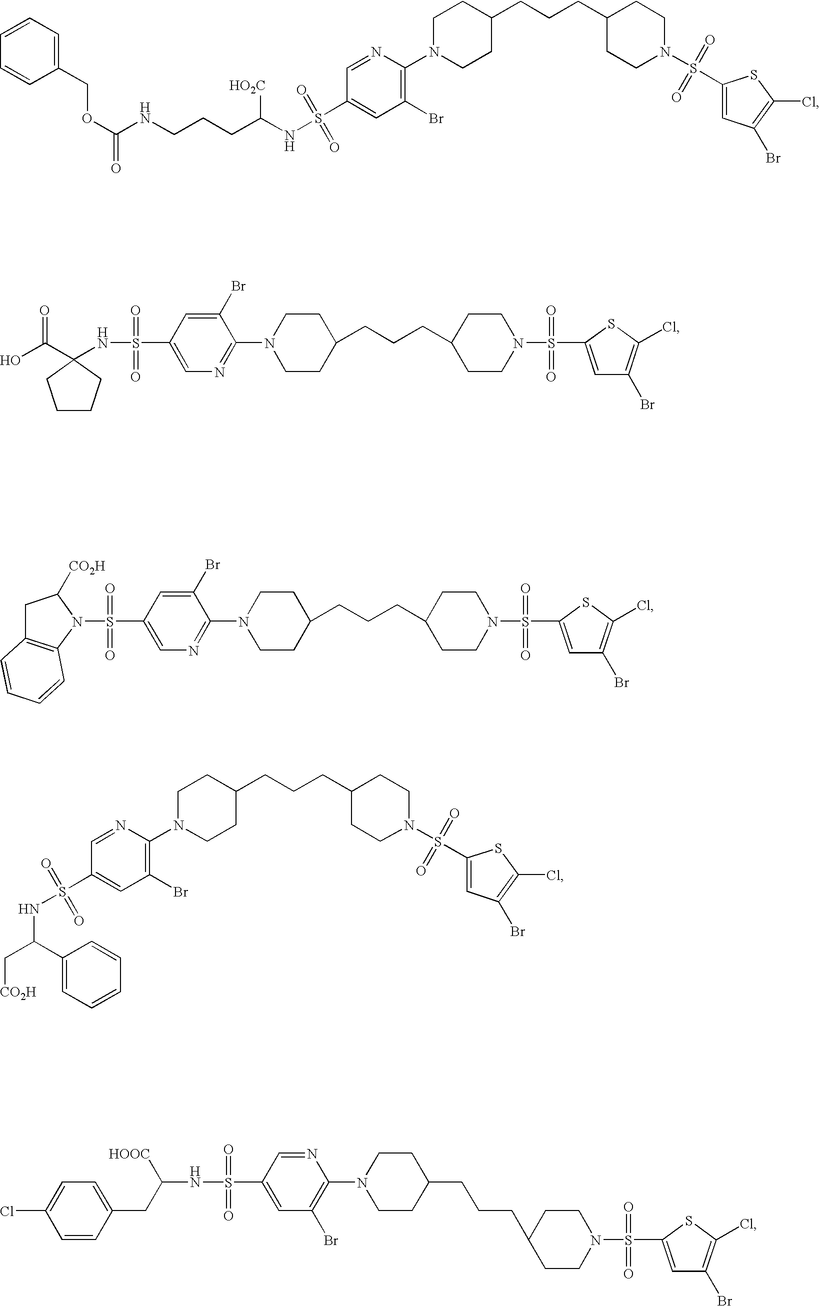

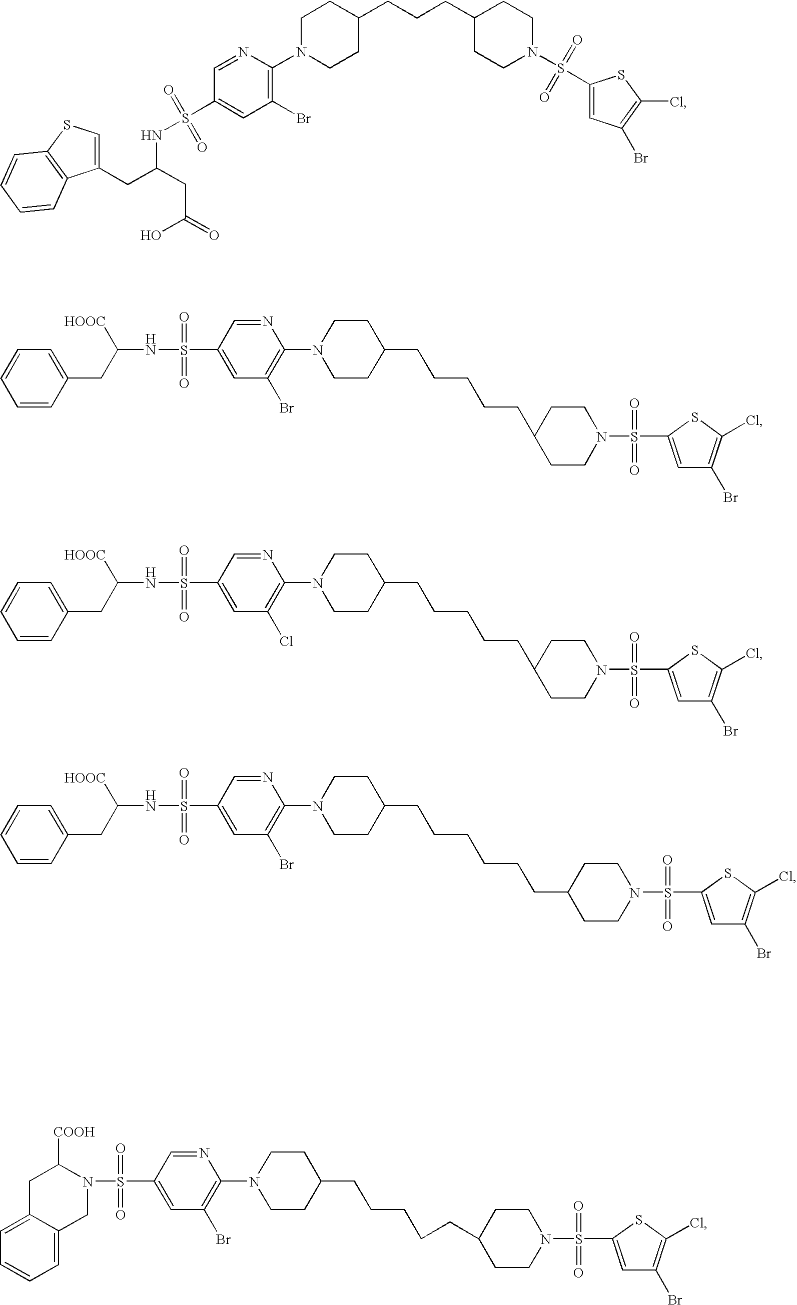

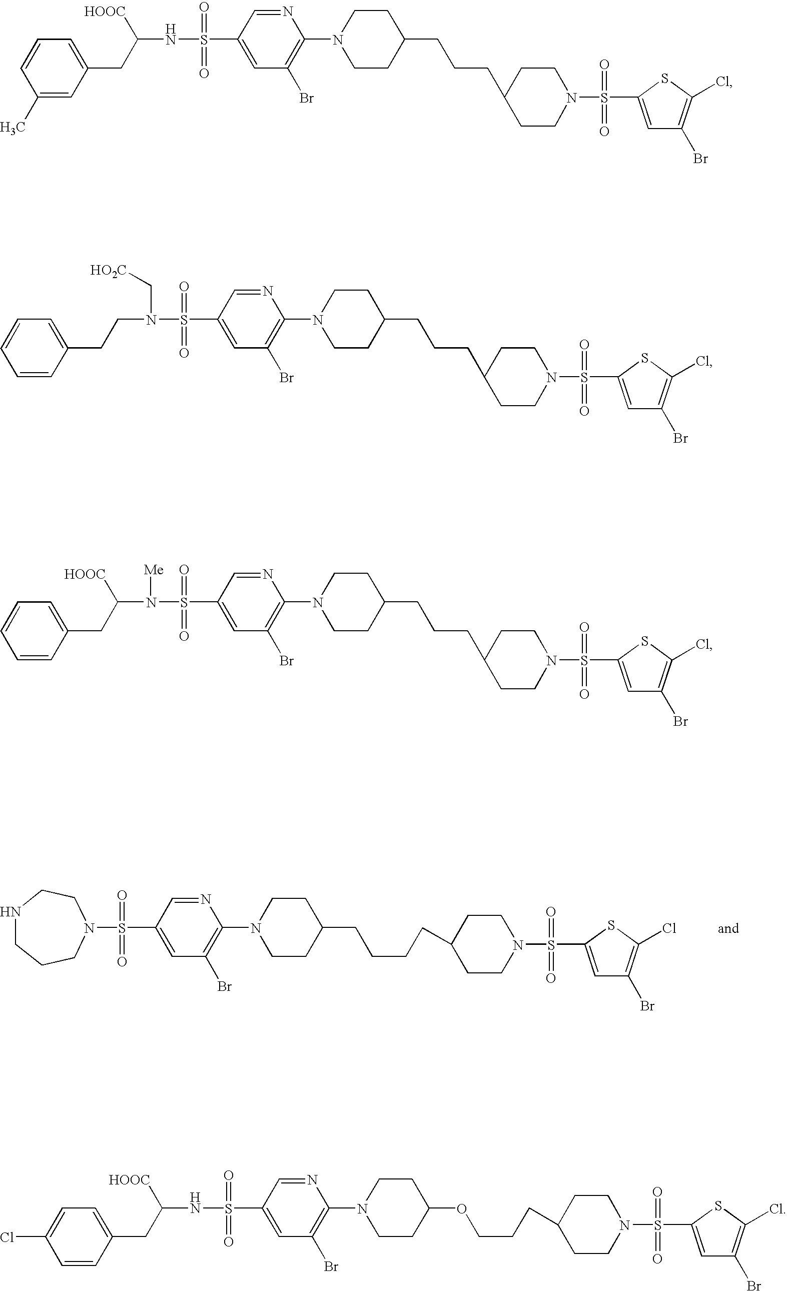

29. A method for treating HIV infectivity, said method comprising administering to a subject in need of such treatment, an effective amount of a compound having formula I: 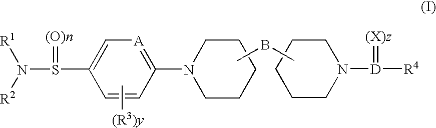
and pharmaceutically acceptable salts thereof;
wherein
R¹ and R² are each independently a member selected from the group consisting of hydrogen, alkyl, cycloalkyl, heterocycloalkyl, arylalkyl, heteroalkyl, hetrocycloalkylalkyl, heteroarylalkyl and —CH₂CO₂H; alternatively R¹ and R² together with the nitrogen atom to which they are attached, form a 4-to 7-membered heterocyclic ring optionally containing 1-3 additional heteroatoms as ring members and optionally substituted with members selected from the group consisting of alkyl, heteroalkyl, aryl, heteroaryl, hydroxy, halogen, —CO₂R¹¹ wherein R¹¹ is hydrogen or (C₁-C₄)alkyl, and alkoxy, wherein if present, any of said substituents located on adjacent atoms in said 4-to 7-membered ring may optionally be replaced with a substituent of formula -E-(CH₂)ᵤ—F— to form a fused ring wherein,
u is an integer from 1-2;
E and F are each independently CH₂, O or NH;
wherein up to three bonds in said fused ring formed may optionally be replaced with a double bond; and
wherein said fused ring formed may further be substituted with 0-4 substitutents selected from the group consisting of halogen, haloalkyl, alkyl, aryl, —CN and —NO₂;
n and z may be the same or different and are integers from 1-2;
R³is a member selected from the group consisting of halogen, alkyl, heteroalkyl, halo(C₁-C₄)alkyl, —S(O)₂R¹³, —S(O)R¹³ and —NO₂, wherein R¹³ is selected from the group consisting of (C₁-C₄)alkyl, alkylamino and amino;
y is an integer from 0-4;
A is N or C;
B is (C₁-C₁₀)alkylene or (C₁-C₁₀)heteroalkylene;
D is C or S;
X, if present, is O, N or S; and
R⁴ is selected from the group consisting of aryl and heteroaryl.

30. The method of claim 29, wherein at least one of R¹ and R² is further substituted with —CO₂R¹⁰, wherein R¹⁰ is (C₁-C₄)alkyl or hydrogen.

31. The compound of claim 29, wherein R⁴ is selected from the group consisting of phenyl, furanyl, benzofuranyl, pyridyl, thienyl and benzothienyl.

32. The method of claim 29, wherein said compound is administered topically.

33. The method of claim 29, wherein said compound having formula I has formula II 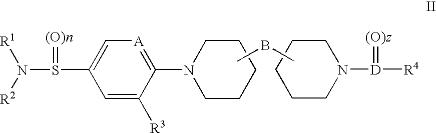
wherein R³ is selected from the group consisting of chloro, bromo and trifluoromethyl.

34. The method of claim 29, wherein [symbol] -NR¹R² is selected from the group consisting of 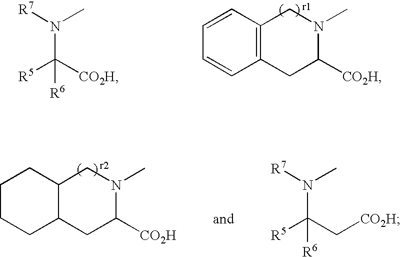
wherein the subscripts r1 and r2 are each an integer from 0-1;
R⁵, R⁶ and R⁷ are independently selected from the group consisting of hydrogen, alkyl, cycloalkyl, heteroalkyl, aryl, heteroaryl, arylalkyl, heteroarylalkyl; or R⁵ and R⁶ together with the carbon atom to which they are attached join to form a 4-to 7-membered ring optionally having heteroatoms as ring members and optionally substituted with members selected from the group consisting of alkyl, heteroalkyl, aryl, heteroaryl, hydroxy, halogen, —C(O)₂H and alkoxy, wherein if present, any of said substituents located on adjacent atoms in said 4-to 7-membered ring may optionally be replaced with a substituent of formula -E-(CH₂)ᵤ—F— to form a fused ring wherein;
u is an integer from 1-2;
E and F are each CH₂;
wherein up to 3 bonds in said fused ring formed may optionally be replaced with a double bond; and
wherein said fused ring formed may further be substituted with 0-4 substituents selected from the group consisting of halogen, halo(C₁-C₄)alkyl, alkyl, —CN and —NO₂.

35. The method of claim 34, wherein [symbol] -NR¹R² is selected from the group consisting of 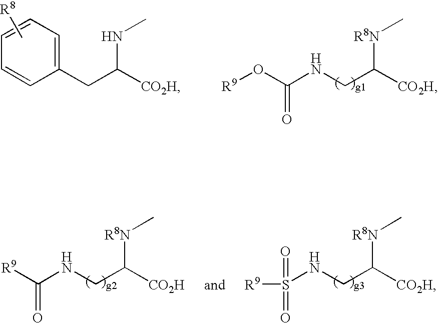
wherein the subscripts g1 to g3 are each an integer from 1-4;
R⁸ is selected from the group consisting of halogen, alkyl, heteroalkyl, aryl, heteroaryl, arylalkyl, heteroarylalkyl, halo(C₁-C₄)alkyl, hydroxyl, alkoxy and —NO₂; and
R⁹ is selected from the group consisting of alkyl, heteroalkyl, aryl, heteroaryl, arylalkyl, heteroarylalkyl and halo(C₁-C₄)alkyl.

36. The method of claim 29, wherein B is (C₂-C₇)alkylene or (C₂-C₇)heteroalkylene.

37. The method of claim 29, wherein A is nitrogen.

38. The method of claim 29, wherein R³ is halogen or halo(C₁-C₄)alkyl; and y is an integer from 1-2.

39. The method of claim 29, wherein z is 2, D is S, and X is O.

40. The method of claim 29, wherein R⁴ is selected from the group consisting of:

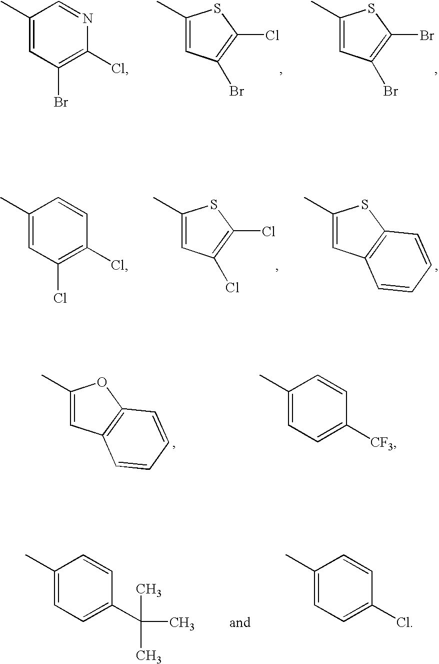

41. The method of claim 29, wherein R⁴ is selected from the group consisting of: 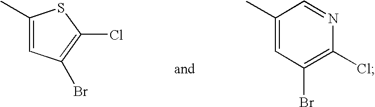
D is S, and z is 2.

42. The method of claim 29, wherein said compound is selected from the group consisting of: 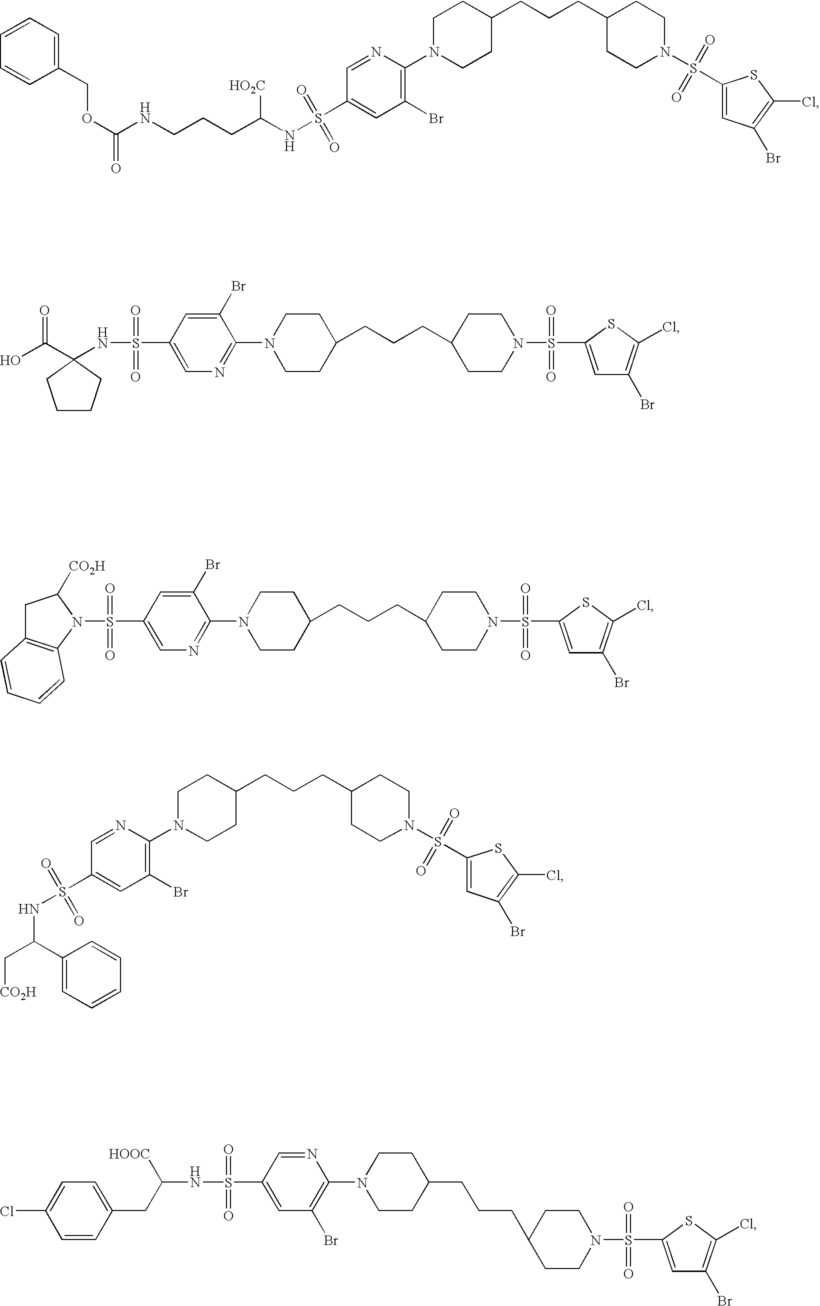 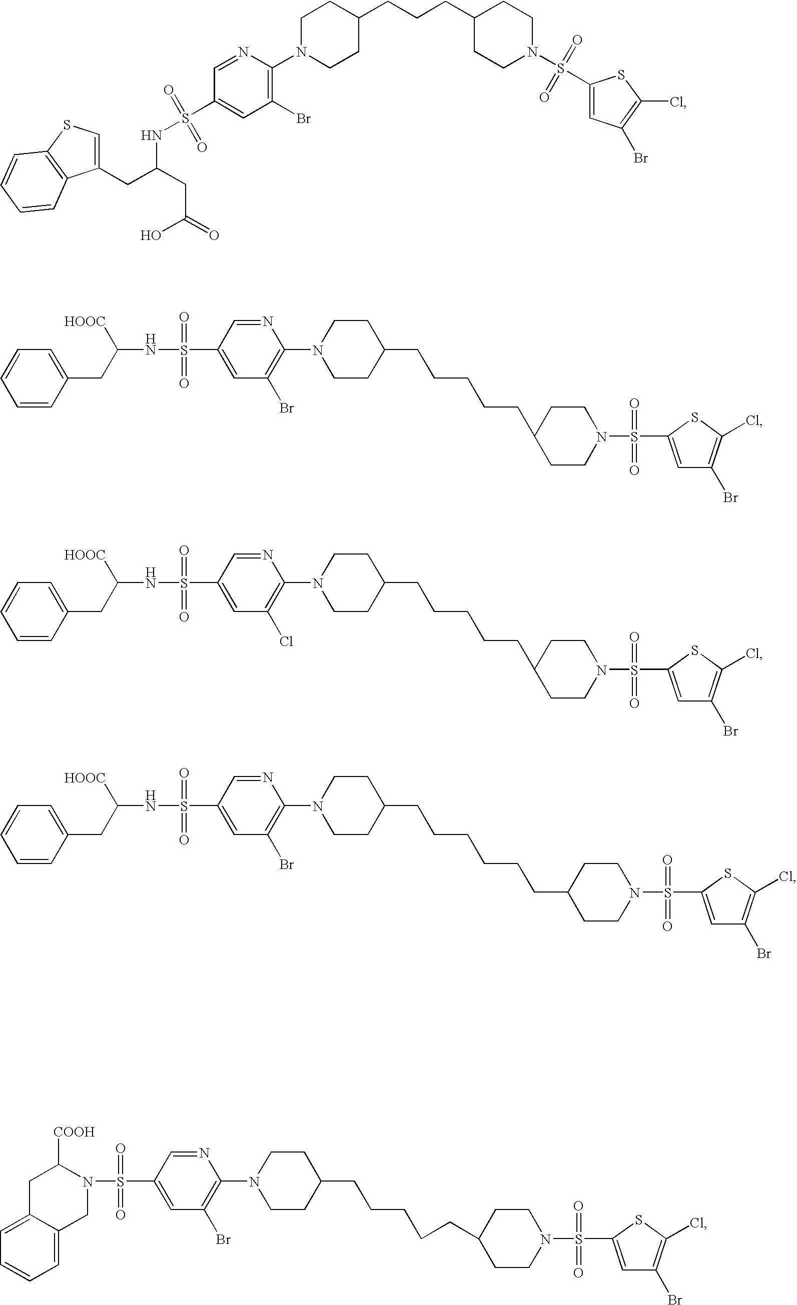 [chemical structure]
